(19) United States
(12) Patent Application Publication
(10) Pub. No.: US 2015/0231276 A1
(43) Pub. Date: Aug. 20, 2015

(54) ROLE OF DH44 AND HOMOLOGS IN CALORIE SENSING

(71) Applicant: New York University, New York, NY (US)

(72) Inventors: Greg Seong-Bae Suh, New York, NY (US); Monica Dus, Ann Arbor, MI (US)

(21) Appl. No.: 14/624,208

(22) Filed: Feb. 17, 2015

Related U.S. Application Data
(60) Provisional application No. 61/940,476, filed on Feb. 16, 2014.

(51) Int. Cl. A61K 49/00

(52) U.S. Cl. CPC A61K 49/0008

(57) ABSTRACT

Provided are methods for identifying Drosophila melanogaster mutants which have mutations affecting calorie sensing behavior. Also provided are methods for identifying agents that can interfere with calorie sensing behavior in D. melanogaster and in mammals. Also provided are methods to identify agents that affect neuronal response of Dh44 neurons or CRF neurons to metabolizing sugars.

### CROSS-REFERENCE TO RELATED APPLICATIONS

This application claims priority to provisional patent application no. 61/940,476 filed Feb. 16, 2014, the disclosure of which is herein incorporated by reference.

### FIELD OF THE DISCLOSURE

This disclosure relates to materials and methods for screening of compounds for their ability to interfere with calorie sensing.

### BACKGROUND OF THE DISCLOSURE

Traditionally it was thought that palatability, through its action on reward centers in the brain, played a major role in reinforcing feeding. However, it has become increasingly clear that calories/nutrients, not palatability, act as primary post-ingestive reinforces on feeding. Animals without taste input sense and respond to the nutritional value of sugars by choosing energy-rich sugars over zero-calorie sweeteners (de Araujio, 2008; Sclafani; Dus, 2011, Miyamoto, 2012). However, the molecular mechanisms involved in sensing the sugars and signaling their nutritional value to determine food choice are largely unknown.

### SUMMARY OF THE DISCLOSURE

We have identified neurons and neural circuitry that underlie caloric sensing using Drosophila melanogaster as a model. The neurons and neural circuitry are important for the post-ingestive effects of nutrient-rich sugars on feeding. We discovered them by genetically manipulating the activity of neuropeptide-secreting neurons in D. melanogaster and tested their effect on food choice behavior. Based on our results, this disclosure provides compositions and methods for identifying agents that can alter food choice behavior.

In one embodiment, this disclosure provides a method of screening Drosophila melanogaster fly mutants to identify mutants with altered calorie sensing behavior comprising: providing a plurality of sets of Drosophila mutant flies, wherein each set comprises a plurality of flies having the same mutation; starving the mutants for a period of at least 5 hours (such as from 5-24 hours); providing access to the flies to a food choice between metabolizing sugar (such as, for example, D-glucose or sucrose) and a non-metabolizing sugar (such as, for example, L-glucose, or sucralose), wherein the food choice comprising the non-metabolizing sugar is at least as sweet as the food choice comprising the metabolizing sugar, and wherein the ingestion of metabolizing and non-metabolizing sugars by the flies can be measurably detected; and if the flies within a set do not show preference to metabolizing sugar over non-metabolizing sugar, then identifying the mutation as affecting calorie sensing behavior.

In one embodiment, this disclosure provides a method of identifying agents that interfere with calorie sensing behavior comprising: providing a plurality of sets of Drosophila melanogaster flies, wherein the flies exhibit a preference for metabolizing sugar under starved conditions; starving the flies for a period of at least 5 hours (such as for 5-24 hours); providing the flies a food choice between metabolizing sugar and non-metabolizing sugar, wherein for each set, at least one test agent is provided with the food choice, wherein the food choice comprising the non-metabolizing sugar is at least as sweet as the food choice comprising the metabolizing sugar; and if the flies within a set do not show preference to metabolizing sugar over non-metabolizing sugar, then identifying the test agent for that set as an agent that affects calorie sensing behavior.

In one embodiment, the screening for identifying agents may be carried out in Dh44 or CRF neurons obtained from the flies or from mammalian sources.

### BRIEF DESCRIPTION OF THE FIGURES

FIG. 1A-1E. Manipulating the activity of Dh44⁺ cells impacts food choice behavior. The food preferences of flies given a choice between a sweet but non-caloric sugar (L-glucose or D-arabinose) and a calorie-rich sugar (D-glucose, trehalose, D-fructose, sorbitol) after 5 h (sated) or 18 h food deprivation (starved). FIGS. 1A-1D) Silencing of the Dh44⁺ cells by expression of the UAS-Kir2.1 transgene using P_{Dh44} GAL4 (blue bar) abolishes preference for the calorie rich sugars in starved flies when compared to genetic controls carrying each transgene alone (gray bars). Flies were given a choice between A) 200 mM L-glucose v. 50 mM D-glucose; FIG. 1B) 200 mM L-glucose v. 100 mM trehalose; FIG. 1C) 80 mM D-arabinose v. 25 mM D-fructose; FIG. 1D) 20 mM D-arabinose v. 20 mM D-arabinose+80 mM sorbitol. FIG. 1E) Activating the DH44⁺ neurons confers starved flies equal preference for both nutritive and non-nutritive sugars. Male flies expressing UAS-NaChBac in the Dh44⁺ neurons using P_{Dh44} GAL4 developed a similar preference for either glucose enantiomer when starved for 18 h (right blue bar), but not when sated (5 h, left blue bar), compared to genetic controls carrying each transgene by itself n=4-10. Statistical analysis was performed by one-way ANOVA with Tukey post-hoc test. ***P<0.0001. Data are the mean+or the mean−SEM for clarity in all graphs.

FIGS. 2A-2E. Dh44+ neurons form a brain-gut axis as their mammalian counterparts. FIG. 2A) The reporter P_{Dh44} GAL4>MCD8GFP (green) labels all the Dh44⁺ cells, visualized by the αDh44 antibody (pink) in a confocal projection image. Scale bar, 10 μm; 1 μm confocal sections. FIG. 2B) Expression of the P_{Dh44} GAL4>mCD8GFP (green) reporter in pars intermedialis of the fly brain counterstained with the neuropil marker nc82 (magenta) in a confocal projection image. Scale bar, 50 μm; 2 μm confocal sections. FIG. 2C) The dendrites of Dh44⁺ cells, visualized by P_{Dh44} GAL4>Dscam-GFP (green), arborize in the suboesophageal ganglion (SOG); Dh44⁺ cell bodies and processes are labeled by the fluorescent protein mko (pink) in a 2D projection of a confocal image. Scale bar, 10 μm; 2 μm confocal sections. FIG. 2D) The neurites of P_{Dh44} GAL4>mCD8GFP cells (bright gray) innervate the fly gut and crop in a fluorescence image. VNC, ventral nerve cord; PV, proventriculus, CB, cell bodies. Scale bar, 100 μm. FIG. 2E) The axons of Dh44⁺ cells, labeled with P_{Dh44} GAL4>Synaptotagmin-GFP (Syt, green) descend along the oesophagus and the PV to innervate the gut and crop in the 2D projection of a confocal image. Scale bar, 20 μm; 2 μm confocal sections.

FIGS. 3A-3G. Dh44⁺ cells are glucosensing and their sugar-mediated activation results in neuropeptide release. The activity of Dh44⁺ cells in response to different sugars was quantified ex vivo in the brains of P_{Dh44} GAL4>UAS-GCaMP3.0 flies. FIG. 3A) Perfusion of 20 mM D-glucose resulted in robust activation of the Dh44^{|} cells compared to the pre-stimulation solution (AHL plus sucrose to balance osmolarity). FIG. 3B) Stimulation with metabolizable sugars, D-glucose (blue bars, 5, 20, 80 mM), trehalose (green), and fructose (magenta) resulted in robust and persistent activation of Dh44⁻ cells with sustained oscillations. Addition of the Na⁺ channel blocker TTX to the D-glucose solution (bright blue bar) had a small but non-significant effect on glucose-mediated activity. n=9-27 brains. FIG. 3C) Representative activity traces of Dh44⁺ cells perfused with different sugars. FIG. 3D) Stimulation of Dh44⁺ cells with sugars induced the release of the Dh44 peptide, as quantified by the immunofluorescence analysis of the amount of Dh44 peptide left inside the Dh44⁺ cells after sugar stimulation (see confocal image of representative cells under each bar). n=24-42 cells. FIG. 3E) Activity of Dh44⁺ cells stimulated by the non-metabolized sugars 2-deoxy-D-glucose (2DG) and L-glucose and by D-glucose plus the hexokinase/glucokinase inhibitor Alloxan (4 μM). The peak amplitude, oscillation number and frequency of Dh44⁺ cells activation was greatly reduced or nearly abolished after treatment with 2DG (pink) and L-glucose (lavender); inhibition of the activity of the first enzyme in glycolysis (hexokinase/glucokinase) in the presence of D-glucose greatly suppressed sugar-mediated Ca⁺⁺ influx (azure bar). n=5-19 brains. ***P<0.0001. FIG. 3F) Stimulation of Dh44⁺ cells with the nonmetabolized sugars 2DG and L-glucose or with D-glucose+ Alloxan (Allx) ex vivo did not result in the release of the Dh44 peptide. n=22-43 cells. **p<0.001. FIG. 3G) Genetic knockdown of the glucokinase homologue Hex-C in Dh44^{|} cells impairs food choice. Food preference of flies expressing an RNAi transgene against different hexokinase/glucokinase genes in Dh44⁺ cells using P_{Dh44} GAL4 when flies were given a choice between 50 mM D-glucose v. 200 mM L-glucose at 18 h-starvation. Control is P_{Dh44} GAL4/+. n=4-6, ***P<0.0001. Statistical analysis was performed by one-way ANOVA with Tukey post-hoc test.

FIGS. 4A-4I. Dh44 signaling regulates the choice for nutritive sugars. The food preferences of flies given a choice between 50 mM D-glucose v. 200 mM L-glucose in the two-choice assay after 18 h food deprivation. FIG. 4A) Alignment of the mature peptide sequence of the human and fly paralogue peptides, human CRF and fly Dh44. FIG. 4B) 18 h-starved flies carrying mutant alleles for Dh44 gene (Mi^{Dh44} or Mi^{Dh44/Def}) failed to choose the metabolized sugar compared to control (w1118^{CS}). Correct food choice was restored in flies where the Mi^{Dh44} mutation was rescued by expression of a UAS-Dh44 construct with the P_{Dh44} GAL4 driver. n=3-8. FIG. 4C) Expression of a Dh44^{RNAi} transgene in Dh44⁺ cells by the P_{Dh44} GAL4 driver (light blue bar) impaired food choice compared to single transgenic controls (gray bars). n=2. FIG. 4D-4I) Signaling downstream of Dh44 mediates food choice. FIG. 4D) Flies expressing mutant alleles for the DH44R1 (Mi^{Dh44R1} and Mi^{Dh44R1/Def}, purple bars) did not prefer D-glucose when starved. n=3-9. FIG. 4E) Silencing of the Dh44R1⁺ neurons by expression of the UAS-Kir2.1 with the P_{Dh44R1} GAL4 impaired food preference in starved flies (purple bar) when compared to single transgenic controls (gray bars). n=4-7. FIG. 4F) Expression of the P_{Dh44R1} GAL4>MCD8GFP transgenes in ten cells in a confocal projection of the fly brain counterstained with the neuropil marker nc82 (magenta). No expression was observed in the gut or the peripheral chemosensory organs. Scale bar, 50 μm, 2 μm sections. FIG. 4G) Mutations in Dh44R2 (Mi^{Dh44R2}, Mi^{Dh44R2/Def1}, and Mi^{Dh44R2/Def2}, pink bars) impaired food choice in starved flies when compared to singly transgenic controls (gray bars). n=3-9. FIG. 4H) Non-conditional or conditional (expressing P_{tubulin} GAL80) ablation of Dh44R2⁺ cells in 18 h-starved P_{Dh44R2} GAL4>Rpr, Hid flies (pink bars) resulted in the incorrect food choice when compared to single transgenic controls (gray bars). n=3-10. FIG. 4I) Expression of the P_{Dh44R1} GAL4>mCD8GFP transgene in a confocal projection of the fly gut counterstained with Phalloidin (pink) and TOPRO (DNA, cyan). No expression was observed in the brain or the external chemosensory organs. Scale bar, sections 2 nm. Inset Magnification of expression of P_{Dh44R1} GAL4>mCD8GFP in a subset of enteroendocrine cells in the midgut. *** P<0.0001. Statistical analysis was performed by one-way ANOVA with Tukey or Dunnet post-hoc tests.

FIGS. 5A-5H. Dh44-mediated activation of its downstream receptor pathways mediates a positive feedback on proboscis extension and gut motor function. FIG. 5A) Food choice of 5 h (sated) or 18 h (starved) food deprived flies given a choice between 200 mM L-glucose v. 50 mM D-glucose in the two-choice assay when Dh44R1⁺ cells were activated by expression of UAS-NaChBac transgene. n=4-10. FIGS. 5B-5D) Acute, temperature-mediated activation of Dh44R1⁺ neurons in P_{Dh44R1} GAL4>TrpA1 flies (lavender bars) resulted in FIG. 5B) a robust proboscis extension response compared to transgenic controls (gray bars), n=12-15; FIG. 5C) an increase in the number of excretions when measured in individual flies (C), n=13-15, or in a population of 20 flies (D), n=3 plates. FIG. 5E-H) Gut motility in response to exposure of w1118^{CS} (FIGS. 5E, 5F) or Mi^{Dh44R2} guts (FIGS. 5G, 5H) to AHL saline (FIGS. 5E, 5G, gray bars) or 10⁻⁶ μm Dh44 peptide in AHL (FIGS. 5F, 5H, light blue bars) after 1 or 6 minutes. Application of the Dh44 peptide results in increased gut motility in controls but not Mi^{Dh44R2} mutant flies. n=9-17. *** P<0.0001. Statistical analysis was performed by one-way ANOVA with Tukey or Dunnet post-hoc tests.

FIG. 6. Feeding results in the release of the Dh44 peptide in vivo. The brains of w1118^{CS} flies fed agar+400 mM L-glucose (gray bar) or agar+400 mM D-glucose (light blue bar) for 45′ minutes were dissected, fixed, and stained for the Dh44 peptide. The amount of Dh44 peptide left in the cells was quantified by measuring the fluorescence intensity of Dh44+ cells labeled with the αDh44 antibody; data was normalized to control (agar+L-glucose). n=24-42 cells. *** P<0.0001. Statistical analysis was performed by Student t test.

FIG. 7. Prandial rise in glycemia is rapid. 18 h food-deprived male w1118^{CS} flies were fed for 5′ 100 mM D-glucose and their hemolymph was collected at different time points after feeding and used to measure the amount of circulating glucose and trehalose. Data was normalized to starved flies for clarity. n=6-11.

FIG. 8. Glycemia and glycogen in Dh44 mutant animals. The levels of circulating glucose and trehalose (top) and glycogen stores (bottom) of control (w1118^{CS}) and Mi^{Dh44} mutant flies were measured at different time points after food deprivation; data is normalized to each genotype 5 h (sated) controls for clarity. n=12-15.

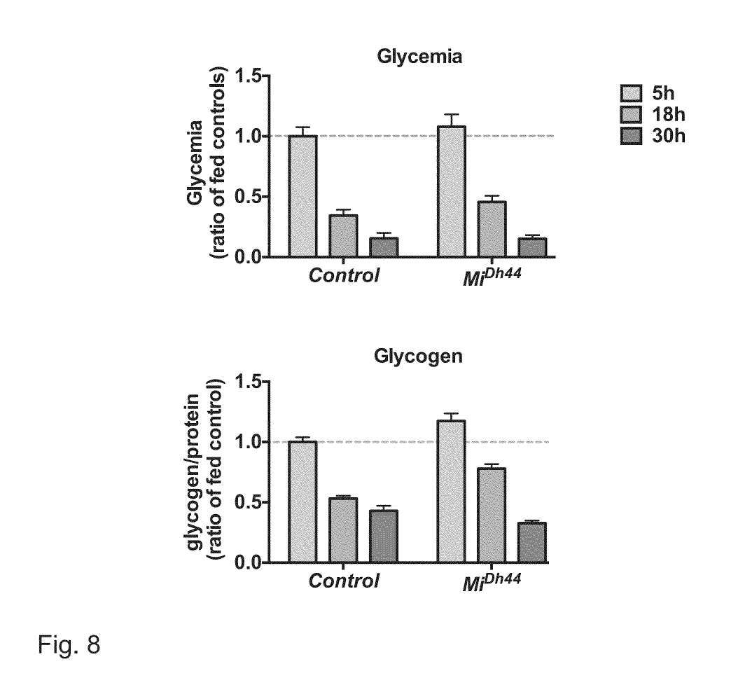

FIG. 9. Silencing or ablation of the Dh44⁺ cells and their downstream pathways does not have an effect on food intake. Thirty 18 h-food deprived males or females flies were fed a mixture of 50% fly food+50 mM D-glucose containing 5% dye (erioglaucine) for 30′. Flies were then flash-frozen, ground in PBS and the amount of food ingested was quantified by measuring light absorption of erioglaucine at 623 nm. Amount eaten is represented as a population average, not for individual flies. There is no significant difference in the amount of food ingested among different genotypes. n=4-6.

FIGS. 10A and 10B are representations of whole-cell patch-clamp recordings from corticotrophin releasing hormone (CRH) neurons in paraventricular nucleus (PVN) in brain slices from mice when exposed to metabolizing sugar D-glucose. FIG. 10A is a continuous recording and shows the electrical activity of the CRH neurons when glucose was changed from 5 mM to 2.5 mM to 5 mM. FIG. 10B shows the data as current step for the same. The electrical activity can be seen to decrease when glucose is reduced from 5 mM to 2.5 mM and then increases when glucose is increased back to 5 mM.

FIGS. 11A and 11B are representations of whole-cell patch-clamp recordings from corticotrophin releasing hormone (CRH) neurons in paraventricular nucleus (PVN) in brain slices from mice when exposed to metabolizing sugar D-glucose. FIG. 11A is the reverse of FIG. 10A. FIG. 11A is a continuous recording and shows the electrical activity of the CRH neurons when glucose was changed from 2.5 mM to 5 mM to 2.5 mM. The electrical activity can be seen to increase when glucose is increased from 2.5 mM to 5 mM and then decreases when glucose is decreased back to 2.5 mM.

### DETAILED DESCRIPTION OF THE DISCLOSURE

The present disclosure provides compositions and methods for identifying agents that can interfere with a caloric sensing pathway involving Dh44 (in Drosophila) or its human homolog, corticotrophin releasing factor (CRF or CRH) or homologs in other species.

The present disclosure provides results showing that specific neurons in the brain, which secrete Dh44 peptide are responsible for caloric sensing function. These neurons are termed as Dh44⁺ neurons in this disclosure. These neurons can also be identified by using antibody against Dh44 or by using endogenous Dh44 promoter to drive the expression of an exogenous marker (such as a fluorescent protein).

The term “wild type” refers to Drosophila having a genome that has not been genetically modified or manipulated in a laboratory such as by recombinant techniques.

The term “Drosophila” as used herein refers to fruit flies Drosophila melanogaster. Drosophila may be of any developmental stage including embryos or eggs, larvae, pupae, and adult flies of any age.

The term “metabolizing sugar” as used herein means any mono or disaccharide that is metabolized within the body. These may also be referred to herein as caloric sugars. Examples include D-hexoses such as D-glucose, D-fructose and the like, and other D-monosachharides. Also included are disaccharides such as trehalose, sucrose and the like. The term “non-metabolizing sugar” as used herein means mono or disaccharides, or derivatives thereof that are not metabolized in the body and provide no caloric value. Examples include L-glucose, 2-deoxy-glucose, arabinose, sucralose, and the like.

Based on the data provided herein, normal caloric feeding behavior (as seen in wild type flies) in D. melanogaster was identified as follows. The metabolizing sugars and non-metabolizing sugars are present in food compositions suitable for D. melanogaster. In general, it is expected that: i) if the concentration of the metabolizing sugar (such as D-glucose) and the non-metabolizing sugar (such as L-glucose) is the same and the flies are not starved, they will show no preference for either; ii) if the concentration of the metabolizing sugar and the non-metabolizing sugar is the same and the flies are starved, they will show a preference for metabolizing sugar; iii) if the non-metabolizing sugar food composition is sweeter (either because the sugar is sweet (as in sucralose versus sucrose) or because it is at a higher concentration (as in higher concentration of L-glucose versus D-glucose)) than the metabolizing sugar food composition and the flies are not starved, they will show a preference for the non-metabolizing sugar; and iv) if the non-metabolizing sugar food composition is sweeter (either because it is sweet (as in sucralose versus sucrose) or because the non-metabolizing sugar is at a higher concentration (as in higher concentration of L-glucose versus D-glucose)) than the metabolizing sugar food composition and the flies are starved, they will show a preference for the metabolizing sugar. One or more of the above observations are termed herein as normal calorie sensing behavior.

In one embodiment, the present disclosure provides methods and compositions for screening drosophila mutants for identifying mutants that do not exhibit preference for metabolizing sugars when starved (i.e., do not exhibit normal calorie sensing behavior). Generation of Drosophila mutants is well known in the art. Screening of mutants can be carried out by starving the flies for various periods of time, and then giving them a food choice of metabolizing or non-metabolizing sugar molecules. A detectable molecule such a detectably colored molecule (for example a food dye) may be included as an indication of food consumption. In one embodiment, the metabolizing or non-metabolizing sugar may itself be detectably labeled. Ingestion of these sugars can be detected by drawing their hemolymph from decapitated flies and determining their concentration.

In one embodiment, the non-metabolizing sugar is provided at a higher concentration than the metabolizing sugar during the food choice step.

In one embodiment, the flies are provided a food choice between food compositions having different degrees of sweetness. The difference in sweetness of the two food choices may be due to a difference in the concentrations of sugars of about equal sweetness or may be due to a difference in the sweetness of sugars. For example, a higher concentration of L-glucose is sweeter than a lower concentration of D-glucose. Alternatively, sucralose, even at the same concentration, is sweeter than sucrose. In one embodiment, the non-metabolizing sugar is present as 2-10 (and all integers therebetween) times higher concentration than the metabolizing sugar. In one embodiment, the non-metabolizing sugar is present 1.5, 2, 2.5, 3, 3.5, 4, 4.5, 5 to 10 times higher than the metabolizing sugar.

The flies may be starved for at least 5 hours. In one embodiment, the flies may be starved for from 5 to 24 hours and all units of time therebetween. In one embodiment, the flies may be starved for from 24 to 48 hours and all units of time therebetween.

In one embodiment, this disclosure provides a method of screening Drosophila melanogaster fly mutants to identify mutants with altered calorie sensing behavior. The method comprises: providing a plurality of sets of Drosophila mutant flies, wherein each set comprises a plurality of flies having the same mutation; starving the mutants for a period of at least 5 hours (such as for any length of time from 5 to 24 hours); providing the flies access to a food choice between metabolizing sugar (such as D-glucose) and non-metabolizing sugar (such as L-glucose), wherein the ingestion of metabolizing and non-metabolizing sugar by the flies can be measurably detected (such as by including a colored additive to the food); and if the flies within a set do not show preference to metabolizing sugar over non-metabolizing sugar, then identifying the mutation as affecting calorie sensing behavior. In one embodiment, the metabolizing and non-metabolizing sugars may be enantiomers. For example, the non-metabolizing sugar may be L-glucose and the metabolizing sugar may be D-glucose. In one embodiment, the metabolizing sugar may be present at a lower concentration than the non-metabolizing sugar. In one embodiment, the non-metabolizing sugar may be sweeter than the metabolizing sugar. In one embodiment, the Dh44 gene in D. melanogaster is replaced by a mammalian homolog. In one embodiment, the mammalian homolog is the human homolog, corticotrophin releasing hormone gene. The ingestion of food may be detected in the hemolymph—as described further in the examples.

In one embodiment, the present disclosure provides methods and compositions for screening test agents that interfere with normal calorie sensing behavior of drosophila flies. In one embodiment, the term “normal calorie sensing behavior” indicates a preference under starved conditions for food choice comprising metabolizing sugar over food choice that may be sweeter, but comprises non-metabolizing sugars. Screening for test agents that interfere with normal calorie sending behavior can be carried out in wild type flies or in mutant flies where the calorie sensing behavior is qualitatively the same as in wild type flies.

In one embodiment, this disclosure provides a method for assessing whether a test agent enhances or, alternatively, diminishes the capability of drosophila to preferentially intake metabolizing sugar (such as D-glucose) when starved. In one embodiment, the assessment is carried out in flies after appropriate periods of starvation (such as from 5 to 24 hours) and all integers therebetween, and administration of the test agent. Their food choice behavior is then assessed as further described herein.

In one embodiment, this disclosure provides a method of identifying agents that interfere with calorie sensing behavior comprising: providing a plurality of sets of Drosophila melanogaster flies, wherein the flies exhibit normal calorie sensing behavior (including a preference for metabolizing sugar under starved conditions); starving the flies for a period of at least 5 hours (such as any length of time from 5 to 24 hours); providing a food choice to the flies between metabolizing sugar (such as D-glucose) and non-metabolizing sugar (such as L-glucose), wherein for each set, at least one test agent is provided with the food choice, wherein the non-metabolizing sugar food choice is sweeter than the metabolizing sugar food choice, and wherein the ingestion of metabolizing and non-metabolizing sugar by the flies can be measurably detected; and if the starved flies within a set do not show preference to metabolizing sugar over non-metabolizing sugar, then identifying the test agent for that set as an agent that affects calorie sensing behavior. In one embodiment, the non-metabolizing sugar food choice is sweeter due to the non-metabolizing sugar being present at a higher concentration than the metabolizing sugar. In one embodiment, the non-metabolizing sugar food choice is sweeter because the non-metabolizing sugar (such as sucralose) is sweeter than the metabolizing sugar (sucrose). In one embodiment, the screening for test agents is carried out in D. melanogaster in which the Dh44 gene is replaced with the human homolog Corticotrophin Releasing Factor gene.

In one embodiment, this disclosure provides a method for identifying agents that affect calorie sensing behavior comprising providing DH44+ neurons of fruit fly; exposing the neurons to metabolizing sugar in the presence or absence of one or more test agents; and determining if neuronal response to the metabolizing sugar is affected by the presence of the test agent and if the neuronal response is affected, then identifying the agent as affecting calorie sensing behavior. Non-metabolizing sugar may be used as a control. The Dh44+ neurons may be present as neurons in culture, in isolated brain tissue or slices, or in the whole animals or flies. Thus, in one embodiment, the effect of metabolizing sugars is tested on the Dh44+ neurons and their response to the metabolizing sugar recorded. The neurons can then be exposed to various test agents in the presence of the metabolizing sugars to determine which agents interfere with the calorie sending behavior. The neuronal response may be in the form of increased electrical activity or change in detectable calcium levels or increased release of certain markers (such as, for example, Dh44 peptide). Electrical activity may be detected using standard patch clamp techniques including whole-cell patch clamp and the like. In one embodiment, activity of neurons can be monitored by calcium imaging technology in conjunction with two-photon microscopy. For example, neuronal activity is measured by monitoring the intracellular calcium levels, which serve as an indicator of neuronal activity. Measurement of calcium levels in neurons is well known in the art.

In another embodiment, the Dh44 neurons can be cultured and agents can be tested by contacting with the neurons (such as by adding to the culture medium) or by depositing directly on to the neurons using micro-pipettes and the like. Release of Dh44 from the neurons can by evaluated to determine if the agent affects the activity of the neurons. For example, the amount of Dh44 released can be determined by using a binding partner to the Dh44 peptide—such as an antibody against the Dh44 peptide. Effect of various agents on the activity/function of Dh44 neurons can be carried out in culture. For this, Dh44 neurons can be isolated and used in primary cultures. The brains are removed from flies at suitable times (such as 60-80 hrs after puparium formation). The isolated brains can be incubated with suitable enzymes such as trypsin, papain, collagenase, DNAse or combinations thereof These techniques are known to those skilled in the art. The dissociated cells may be washed with serum-free growth medium. The cells may be mechanically dissociated. Although this can break off the axons and dendrites of differentiated neurons, it is considered that these will regenerate in culture. The presence of Dh44 neurons can be confirmed by immunofluorescence or by detecting the release of Dh44 into the culture medium. The neuronal culture can be maintained in serum-free medium.

In one embodiment, this disclosure provides a method of identifying agents that interfere with the functioning/activity of Dh44 neurons or mammalian CRF neurons comprising the steps of: a) providing a primary culture of Dh44 neurons—either from wild type flies or flies in which Dh44 gene has been replaced by CRF gene, or a primary culture of mammalian CRF neurons, measuring a base level of release of Dh44 peptide or CRF into the culture medium, contacting the neurons with a test agent and measuring if the level of Dh44 or CRF that is released has changed, wherein a change in the release of Dh44 or CRF is indicative that the test agent affects the function/activity of the Dh44 or CRF neurons.

In one embodiment, a method is provided for identifying agents that affect food choice behavior in fruit fly D. melanogaster comprising the steps of administering a test agent to the fly and determining if any of the steps in the Dh44 pathway or CRF pathway are altered.

In certain embodiments, the disclosure includes genetically modified fruit flies and methods of making them. Thus, in certain embodiments, the endogenous D. melanogaster gene Dh44 may be replaced with a mammalian Corticotropin-releasing hormone (CRH) gene, also known as corticotropin-releasing factor (CRF). In certain embodiments, the mammalian CRF gene is a human CRF gene. The sequence of human CRF is provided in SEQ ID NO: 7. Thus, the disclosure includes humanized fruit flies. The DNA sequences of Dh44 and the human CRF gene are known in the art, as are the amino acid sequences of the proteins they encode. Any suitable method for experimentally modifying a fruit fly genome/chromosome can be adapted to make D. melanogaster comprising a replacement of its endogenous Dh44 gene with a mammalian CRF gene, and many such techniques are known in the art. In an embodiment, the disclosure includes making a targeted replacement of D. melanogaster Dh44 using a segment of DNA comprising a human CRF gene to replace the Dh44 gene. In embodiments, the Dh44 gene can be replaced with a human CRF gene with approaches which include but are not necessarily limited to homologous recombination, P element-induced gap repair, a phiC31 integration system, site-specific integrase-mediated repeated targeting (SIRT) and long range SIRT, 1- or 2-step captured segment exchange approaches, Ends-In Gene Targeting or Ends-Out Gene Targeting approaches, or any other suitable methods.

Using the humanized flies, potential inhibitors of CRF may be tested for any effects (diminishing or enhancing) on food choice behavior such as preference for metabolizing sugar (such as D-glucose over a higher concentration of L-glucose) in starved flies.

Similarly, humanized flies in which Dh44 receptors are replaced with CRF receptors can also be prepared. Such humanized flies could be used for identifying inhibitors of Dh44 activity/function via inhibition of the receptors. Such flies could also be used for identifying inhibitors or stimulators of gut motility.

In certain embodiments, brain slices from other non-human animals (such as mice) could be used to screen for agents that interfere with normal neuronal response of CRF neurons. Metabolizing sugar may be directly added to the culture medium in which the brain slices are placed. The effect on the electrical activity of the CRF neurons can be recorded first as a reference control and then upon the addition of a test agent to determine the effect of the test agent on the response of neurons to metabolizing sugar.

In one embodiment, the wild type flies or the humanized flies may be used to identify agents that may inhibit or stimulate gut motility.

The following examples are provided to further describe the invention. The examples are intended to be illustrative and not restrictive.

### EXAMPLE 1

### Material and Methods

### Fly Strains

Flies were grown in standard cornmeal-molasses medium at low density at 25° C. w1118 flies backcrossed to Canton-S (CS) 10 times, referred to herein as w1118^{CS}.

### Transgenic Lines

P_{Dh44} GAL4 was generated by cloning an ˜800bp region upstream of the Dh44 gene promoter into pCasper4-AUG-GAL4X. P_{Dh44R1} GAL4 and P_{Dh44R2} were generated in the same way by cloning the ˜1 kb fragment upstream of the Dh44R1 and Dh44R2 genes into pCasper4-AUG-GAL4X. GAL4 Transgenic flies were generated by Bestgene, Inc.

### Two-Choice Assay

Feeding assays were carried out as previously described (Dus et al, PNAS, 2011; Dus et al. Nature neuroscience, 2013). Briefly, 35 4-8 days old male flies were food deprived in an empty vial with a Kim wipe wetted with 2 ml of MilliQ for 5 h or 18 h, and then given a choice between two sugars each color-coded with a tasteless food dye for 2 hours. Food preference was scored as percent preference index (% PI) by scoring the abdomen color of each fly:

% PI=(#ate food1+0.5*#ate both)−(#ate food2+0.5*ate both)](total # flies ate)

All sugars, except for L-glucose (Carbosynth), were from Sigma. The concentrations of each sugar is indicated in the legends.

### Hemolymph Glycemia and Glycogen Measurements

Glycogen and glycemia were measured as previously described (Dus et al 2011, 2013; Rufilson et al; Xu and Seghal et al). For prandial measurements of hemolymph glycemia, flies were starved for 18 hours, fed with 100 mM for different lengths of times and their hemolymph collected immediately.

### Immunofluorescence

Staining of brains was carried out and gut immunostaining was also performed The flies were fed agar based food for two days to decrease background. Antibodies were as follows: mouse anti-nc82 (1:50; Developmental Studies Hybridoma Bank), rabbit anti-GFP IgG (1:500; no. A11122, Invitrogen) anti mouse-biotin (1:200), rabbit-αDh44. Secondary antibodies were Alexa Fluor 647-Strepavidin (1:500, Invitrogen), Alexa Fluor 488 goat anti-rabbit IgG (1:500, Invitrogen); TO-PRO3 (642/661 nm; 1:500) was used for DNA labeling and Red-Phalloidin (Invitrogen) was used for gut labeling. Images were acquired with a Zeiss LSM 510, acquisition was at 1-2 μm intervals (refer to legends) with 1024×1024 or 512×512 resolution.

### Destaining Experiments

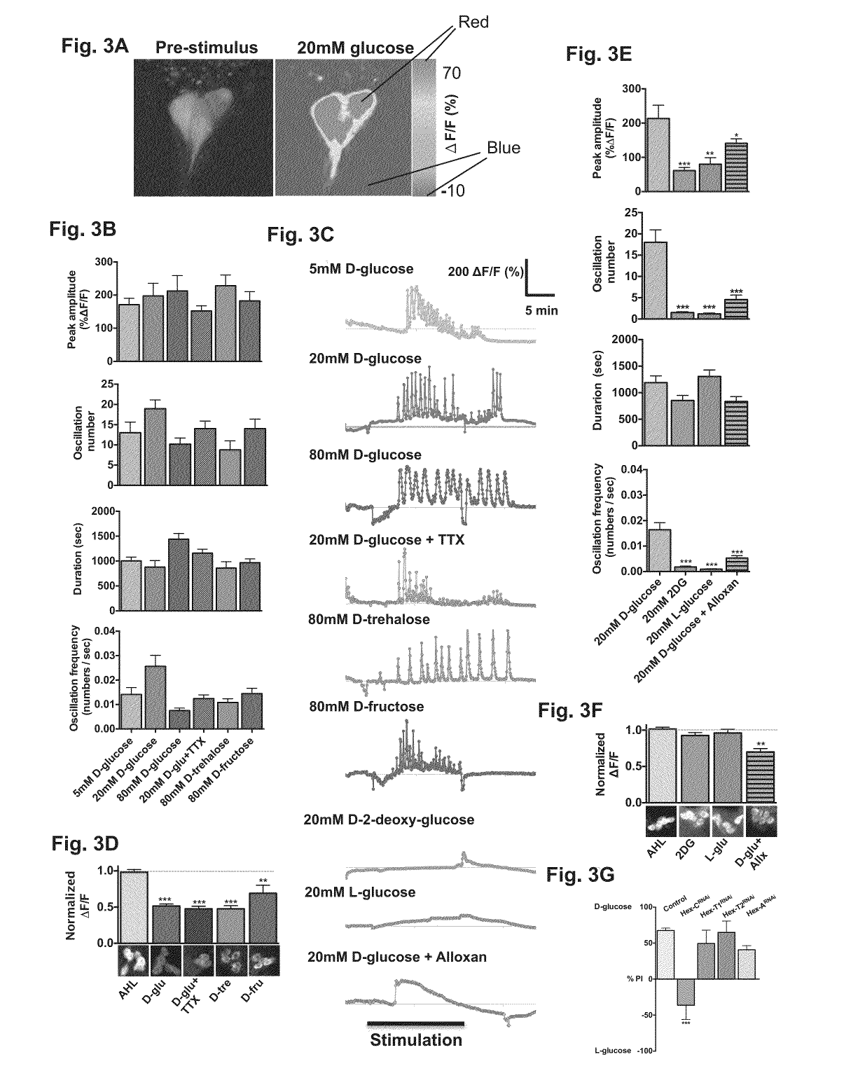

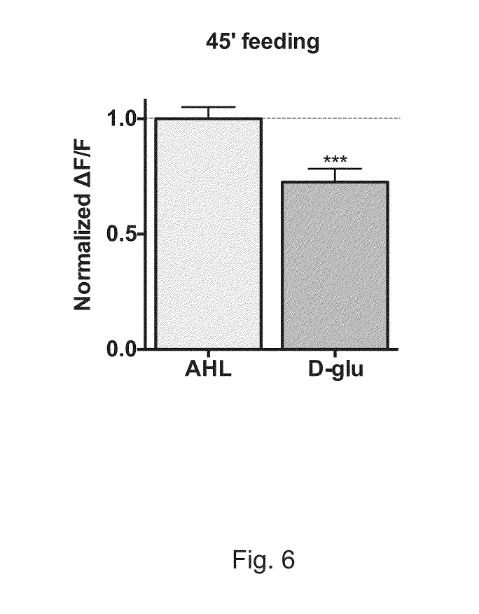

For the ex vivo destaining experiments in FIGS. 3A-3G the brains of 18 h-food deprived flies (males and females) were rapidly dissected in sugar-free AHL, incubated in either AHL saline alone or AHL+ sugar at 80 mM concentration for 30′, immediately fixed and stained with αDh44 antibodies as per immunofluorescence protocol above. Image acquisition was done using a Zeiss LSM 510 confocal microscope with fixed laser and gain setting between samples. ImageJ was used to calculate the fluorescence intensity/cell. For in vivo destaining experiments (FIG. 6) flies were fed 400 mM L-glucose+agar or 400 mM D-glucose+agar for 45′, after which brains were dissected and treated for standard immunostaining with αDh44 antibodies. Image acquisition and analysis was done as for ex vivo destaining experiments.

### Gut Motility Measurements

One fly gut at the time was rapidly but gently dissected in AHL with attention not to disrupt the attached tissues, keeping the head intact and removing the cuticle, muscles and fat. The exposed gut was pinned onto a Sylgard plate with fine tungsten pins through the proboscis and a small piece of cuticle attached to the last part of the gut, and bathed in ˜13 μl of AHL confined by ring-binder protectors. Each gut was imaged with a Zeiss High-speed camera (2 frames/sec) connected to a stereomicroscope with a 0.6× magnification. After 5′ in AHL, the solution was removed by capillary action and replaced with 13 μl AHL+10⁻⁶ μm Dh44 peptide or AHL alone and video acquisition rapidly restarted (less than 10″) for 10′. Each video was processed with the Zeiss AxioPlan 4.8 software and converted into an AVI file with 7 frames/second. Quantification of gut contractions was done visually beginning counting one minute after addition of the solution to avoid diffusion artifacts: AHL+1′ for 4′, Dh44+1′ for 4′, and Dh44+5′ for 4′ (in the avi files, 1′ of real-time image acquisition corresponds to about 15″). Data for each gut was normalized to the initial AHL alone condition.

### PER Measurements

A single fly was gently trapped into a p200 tip cut so to expose the fly head and forelegs comfortably. Each tip was inserted perpendicularly onto a microscope slide covered with clay and placed at the bottom of a stereomicroscope in a room heated to 30° C. After 5′ each fly was observed through the objective of the microscope and proboscis extensions counted and scored per minute. To acquire a video of PER, flies were gently trapped into a glass Pasteur pipette with a small cotton plug, transferred to a 30° C. heated room for 5′ minutes and video capture was done using a Zeiss high-speed camera and stereomicroscope at 2 frames/second for a few minutes.

### Excretion Assays

Single-fly assay: a single fly was gently introduced into a glass Pasteur pipette sealed with a small cotton plug and ˜5 μl water to prevent long-term desiccation and immediately transferred to a 30° C. heated room. The numbers of excretions (well visible against the glass) were counted after 10′, 20, and 60′.

### Population Assays

20 males flies previously fed food+10% blue dye for 3 days were anesthetized in ice, rapidly introduced into a 5 cm plastic Petri dishes containing filter paper and immediately transferred to a 30° C. heated room for 60′. The number of excretions (colored blue) was visually quantified using a stereomicroscope.

### Colorimetric Food Ingestion Assay

30 males or females 18 h-starved flies were given access to food (50% fly food/50 mM D-glucose+0.5% erioglaucine) for 30′, flash-frozen, ground up in 1 ml of PBS, and spun down on a tabletop centrifuge at the maximum speed for 10′. 100 μl of the supernatant was transferred to a plate reader and light absorbance measured at 625 nm. Background (minimal) from flies fed the same food without the dye was subtracted from each reading. The amount of food eaten by the group of flies was linearly regressed from known standards. Background (minimal) from flies fed the same food without the dye was subtracted from each reading.

### Statistics

GraphPad Prism software was used for all graphs and statistical analysis. Data represent multiple independent experiments. Error bars are SEM, and *** p<0.0001. Student-t test or one-way ANOVA were used according to the number of conditions and genotypes and specified in each legend.

### Results

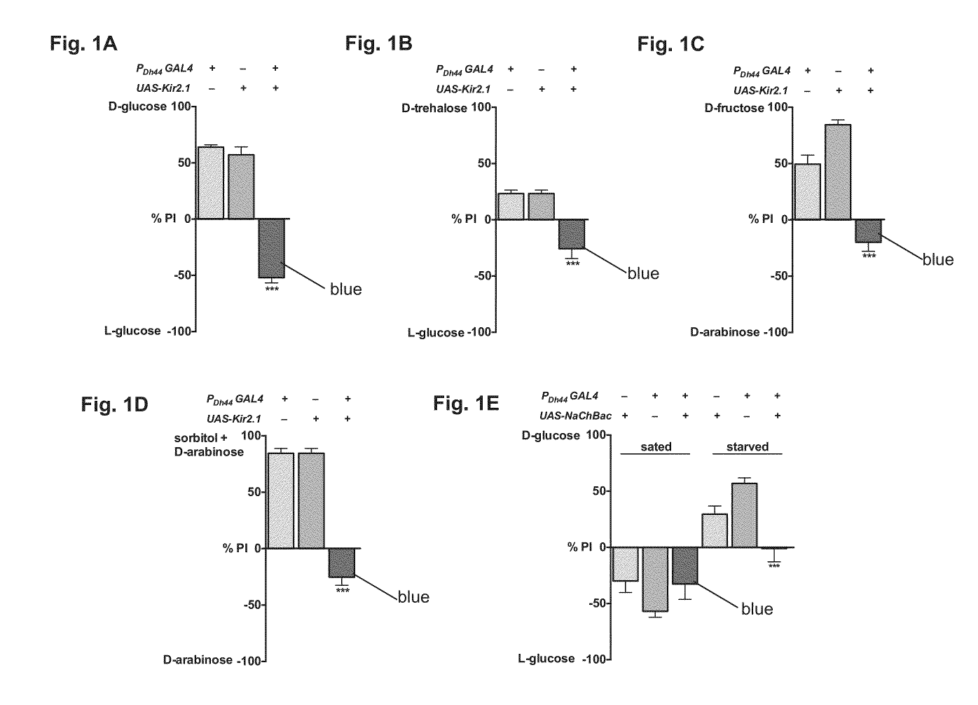

The activity of neuropeptide secreting neurons in Drosophila melanogaster was genetically manipulated and the flies were tested for food choice behavior. In the fly a preference for a less concentrated but calorie-rich sugar (50 mM D-glucose) over a more-concentrated (sweeter) zero-calorie sweetener (200 mM L-glucose) increases gradually with starvation time (Dus, 2013); 18 h starved flies override palatability for energy and choose D-glucose over L-glucose (FIG. 1A, gray bars). However, silencing the neurons expressing the Diuretic hormone 44 neuropeptide, (Dh44, the insect paralogue of the mammalian Corticotropin releasing hormone, CRH), by overexpression of the inwardly rectifying K⁺ channel Kir2.1, abolished the preference for nutrient-rich D-glucose in starved flies (FIG. 1A, blue bar). Flies expressing the P_{Dh44} GAL4>Kir2.1 transgenes failed to respond to the nutritional value of all sugars tested, including the hemolymph sugar trehalose, the hexose D-fructose, and the tasteless but calorie rich sugar-alcohol sorbitol (FIG. 1B, 1C, 1D, blue bars respectively) when given a choice between these sugars and the zero-calorie sweeteners L-glucose or D-arabinose (controls, gray bars), suggesting that Dh44⁺ neurons are necessary for nutrient sensing.

Activation of Dh44⁺ by expression of the depolarizing Bacterial Voltage-gated Na⁺ Channel UAS-NaChBac resulted in an equal choice for both nutritious (D-glucose) and non-nutritious (L-glucose) sugars in starved flies (FIG. 1E, sated flies preferred L-glucose because of palatability). Thus, Dh44⁺ neurons are necessary and sufficient to signal the nutrient-value of sugars, and their activation communicates to the fly the nutritional value of sugars.

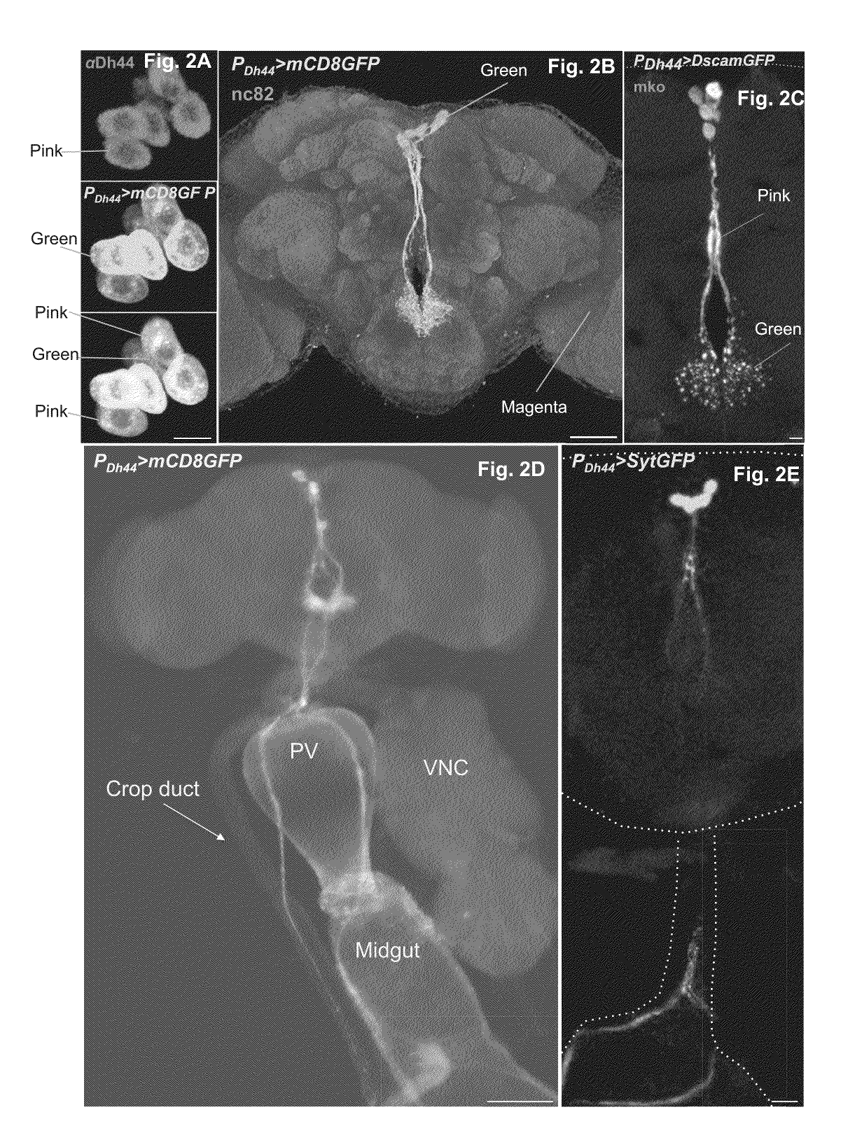

We next focused on the anatomy of the Dh44⁺ neurons. The P_{Dh44} GAL4>GFP transgenes labeled six large cells in the pars intermedialis (FIGS. 2B, and 2D), a region of the fly brain rich in neurosecretory cells, and together with the pars intercelebralis (located above it), thought to be a functional and neuroanatomical correlate of the mammalian hypothalamus. The P_{Dh44} GAL4 reporter faithfully recapitulated the endogenous expression of the Dh44 neuropeptide (FIG. 2A) as revealed by a α-Dh44 antibody. Dh44⁺ neurons have neurites both in the sub-oesophageal ganglion (FIG. 2B, SOG), and in the fly gut (FIG. 2D). We mapped the polarity of the Dh44⁺ neurons by using the florescent pre- and post-synaptic markers, Synaptotagmin-GFP (Syt-GFP) and Down Syndrome cell adhesion molecule-GFP (Dscam-GFP), respectively. The Dh44⁺ neurites in the SOG are dendrites, (FIG. 2C, P_{Dh44} GAL4>Dcam-GFP dendrides in green; Dh44⁺ cells are in magenta, labeled by the monomeric Kusabira orange protein, mko), while the long processes that diverge above the proventriculus (PV) to innervate the gut and crop are axons (FIGS. 2D and 2E, P_{Dh44} GAL4>Syt-GFP axons and cell bodies in green). Because taste information from taste-receptor expressing neurons relays to the SOG, the Dh44^{|} neurons could provide a way to integrate taste input and nutritional information, while providing a link to the gut-brain axis.

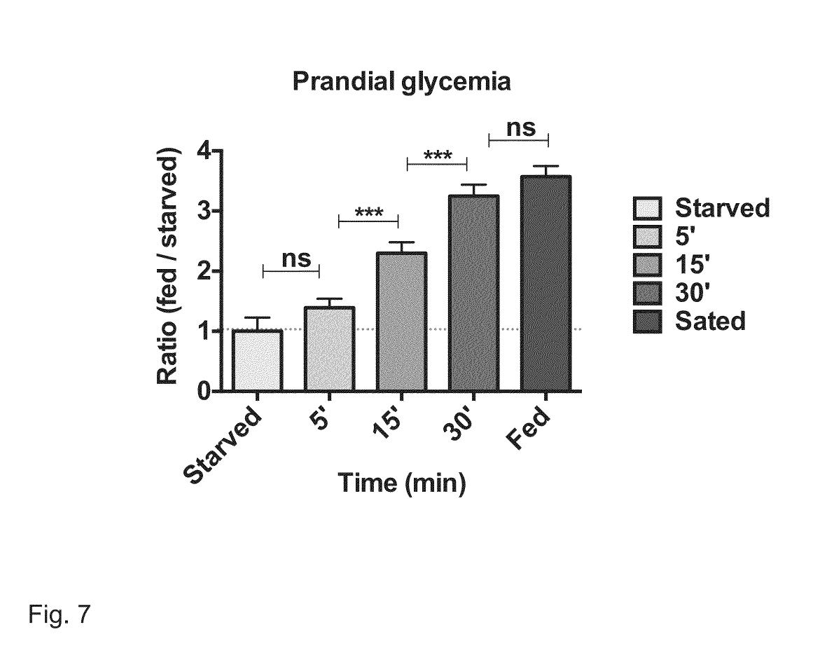

To examine if the Dh44⁺ neurons not only signal, but also directly sense nutritional information we tested their activation by sugars in the brains of flies carrying the fluorescent Ca²⁺ indicator UAS-GCaMP3.0 specifically in the Dh44⁺ neurons (P_{Dh44} GAL4>GCaMP3.0). Perfusion with different concentrations of D-glucose (FIGS. 3A and 3B, top, 5, 20, 80 mM, blue bars), trehalose (green bar) and D-fructose (plum bar) robustly activated the Dh44⁺ cells. The sugar-mediated response was delayed from the onset of sugar stimulation and long lasting (FIG. 3D, compare representative single-cell traces) with discrete oscillations that became less frequent but longer with increasing concentrations of sugars (FIG. 3A, compare oscillation number and frequency). The presence of discrete oscillations is a characteristic feature of neurosecretory cells that is linked to neuropeptide release. We then asked if sugar-mediated activation of the Dh44⁺ cells results in the release of the Dh44 neuropeptide. We incubated fly brains with sugars, fixed and stained them with the α-Dh44 antibody, and quantified the amount of Dh44 peptide left inside the cell bodies. The Dh44⁺ cells of brains treated with sugars (FIG. 3E, blue and green bars) had half of the amount of Dh44 neuropeptide compared to the brains treated with saline alone (FIG. 3E, gray bar, AHL). Thus, sugar activation from ingested foods is responsible for the release of the neuropeptide (FIG. 3D) To test if Dh44⁺ neurons directly respond to sugars we perfused fly brains with glucose together with the voltage-gated Na^{|} channel blocker Tetrodotoxin (TTX). Addition of TTX to D-glucose had a small but nonsignificant effect on the response of Dh44⁺ cells to the hexose (FIG. 3B, electric blue bar), and did not affect neuropeptide release (FIG. 3E), indicating that the Dh44⁺ neurons are directly activated by sugars. Thus, Dh44⁺ neurons are glucosensing; according to our data, sugar ingestion is rapidly followed by an increase in glycemia (FIG. 7), which in turn activated the Dh44+ neurons and triggers neuropeptide release. We next asked if the Dh44⁺ neurons are also activated by non-metabolized sugars. Perfusion of L-glucose (D-glucose enantiomer) or D-2′-deoxy-glucose (D-glucose analog) resulted only in a small increase in activity that lacked oscillations (FIG. 3C, 3D, pink and lavender bars and traces) and did not induce neuropeptide release (FIG. 3F). Therefore, Dh44⁺ neurons only respond to metabolized sugars. This strongly suggests that sugar metabolization, not entry, is the mechanism used by Dh44⁺ to respond to sugars. To test this hypothesis we perfused fly brains with D-glucose+ Alloxan, a widely used inhibitor of hexokinase/glucokinase, (the enzymes that convert glucose into glucose-6-phosphate, the first step in sugar metabolism/glycolysis). This resulted in a dramatic decrease in the peak amplitude of activation, together with an almost complete disappearance of oscillations (FIGS. 3C and 3D, azure bars and cell trace), and a reduction in Dh44 neuropeptide release (FIG. 3F, azure bar). Thus, Dh44⁺ cells are metabolic glucosensing neurons. To address the relationship between glucosensation and preference for nutritious sugars, we expressed RNAi transgenes against different hexokinase/glucokinase enzymes in Dh44⁺ neurons and tested their effect on food preference for D- v. L-glucose in 18 h food-deprived flies. Only knockdown of the mammalian glucokinase homologue, the fly Hexokinase-C (Hex-C), impaired selection of the energy-rich D-glucose. This is intriguing because glucokinase, thanks to its unique kinetic properties, functions with increasing concentrations of glucose in the physiological range without being saturated (unlike other hexokinases). Because of this, glucokinase is widely considered a molecular ‘glucose sensor’ and its activity has been implicated in the physiology of glucosensing cells, most notably the β-cells of the pancreas, but also in a subset of hypothalamic glucosensing neurons. However, how neural glucosensation is linked to feeding behavior is still unclear. Our data on Dh44⁺ cells show that metabolic glucosensation is required for food choice behavior, forging a physiological link between neural glucosensation and feeding. It also provides an explanation on how only nutritive, but not non-nutritive sugars can induce positive post-ingestive effects.

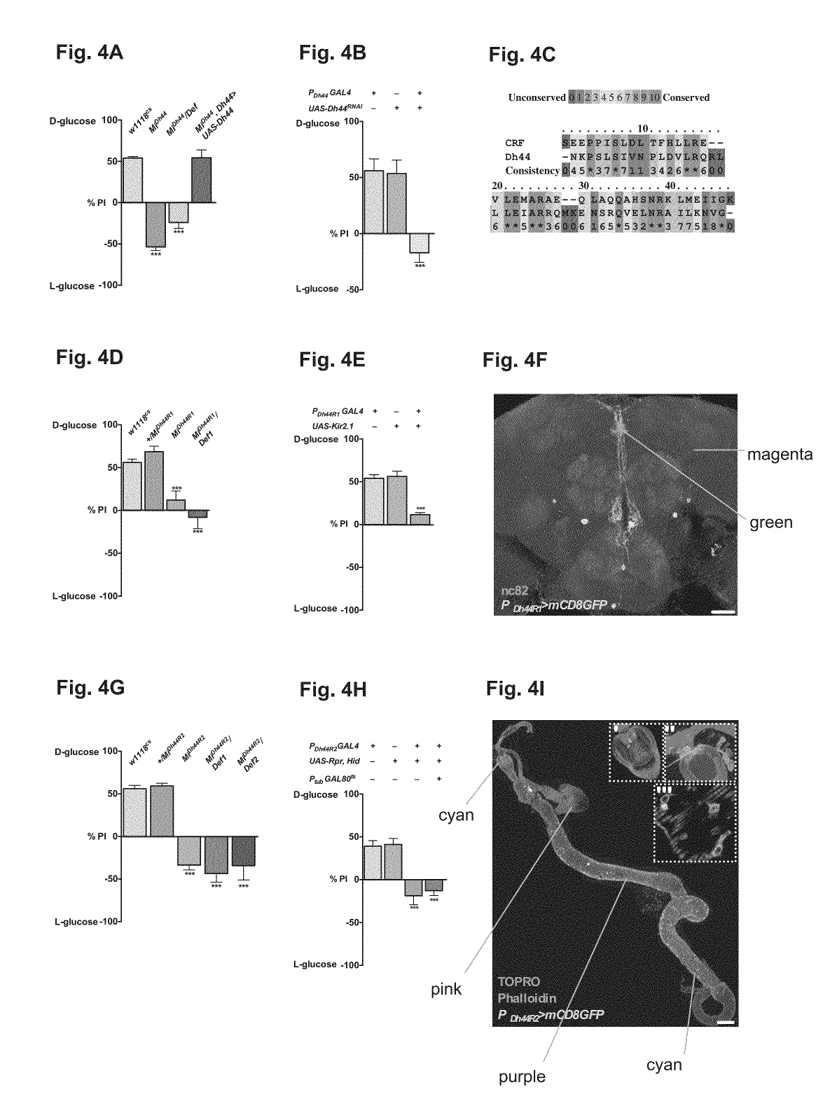

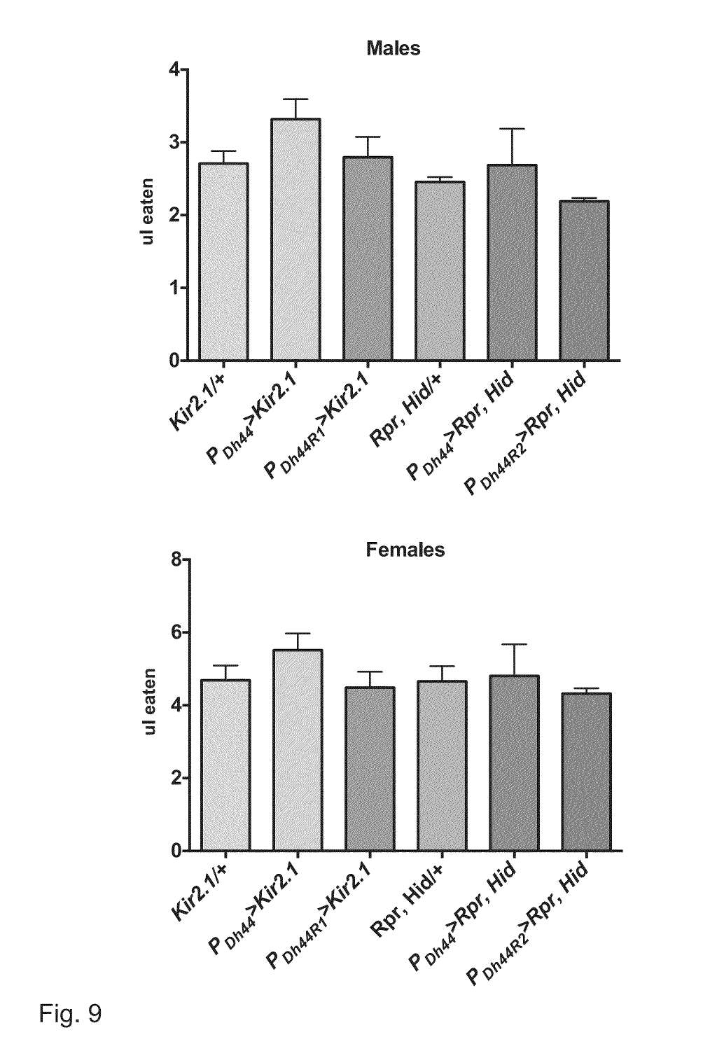

A question that still remains open is how sugar-mediated activation of Dh44⁺ neurons results in a positive behavioral reinforcement for nutritive sugars. To answer this, we focused on the effector mechanisms downstream of nutrient sensing by these neurons. Dh44 neurons release the Dh44 neuropeptide, the fly paralogue of the human CRH (FIG. 4A), in response to sugar activation (FIG. 3E). We asked if this neuropeptide is required for the choice of nutritive sugars in food-deprived flies. Flies carrying mutant alleles of the Dh44 gene or a Dh44^{RNAi} transgene had impaired food choice behavior but normal metabolism (FIGS. 4B and 4C; FIG. 9), which was rescued by expression of a UAS-Dh44 transgene in the Dh44⁺ neurons (FIG. 4B, Mi^{Dh44}; P_{Dh44} GAL4>UAS-Dh44). Therefore, the Dh44 neuropeptide is also required for food choice behavior. In particular, release of Dh44 signals the presence of nutritious sugars, providing an inroad to dissect how signaling downstream of this peptide hormone results in food choice behavior. Flies, like mammals, have two receptors for the Dh44/CRH peptide, the Dh44-Receptor 1 (Dh44R1) and the Dh44-Receptor 2 (Dh44R2), which are activated by Dh44 binding. Flies carrying mutant alleles for each receptor showed impaired food choice for nutritive sugars (FIGS. 4D and 4E, R1 purple bars, R2 pink bars) when given a choice between D- v. L-glucose at 18 h food-deprivation, indicating that both receptors are required. To understand how each receptor contributes to food choice behavior, we generated flies expressing the GAL4 protein under the control of either the R1 (P_{Dh44R1} GAL4) or the R2 (P_{Dh44R2} GAL4) promoter. The P_{Dh44R1} GAL4>GFP reporter was expressed in just ˜10 cells in the fly brain (FIG. 4F), but not in the gut. Conversely, the P_{Dh44R2} GAL4>GFP reporter showed expression in a large number of gut cells (FIG. 4I), among which enteroendocrine cells (FIG. 4I, inset), but not in the brain. Silencing of the R1⁺ neurons with UAS-Kir2.1 (FIG. 4E, P_{Dh44R2} GAL4>Kir2.1) or ablation of the R2⁺ gut cells with the cell death proteins UAS-Rpr and UAS-Hid (FIG. 4H, P_{Dh44R2} GAL4>Rpr, Hid, +/−P_{tubulin}GAL80^{ts} to control induction of Rpr and Hid postnatally) blocked the choice for the nutritive sugar D-glucose over the sweeter, but zero-calorie sweetener L-glucose in food-deprived flies. The fact that the two receptors are spatially segregated but both required for food choice behavior suggests that they likely direct different physiological effects downstream of Dh44 signaling.

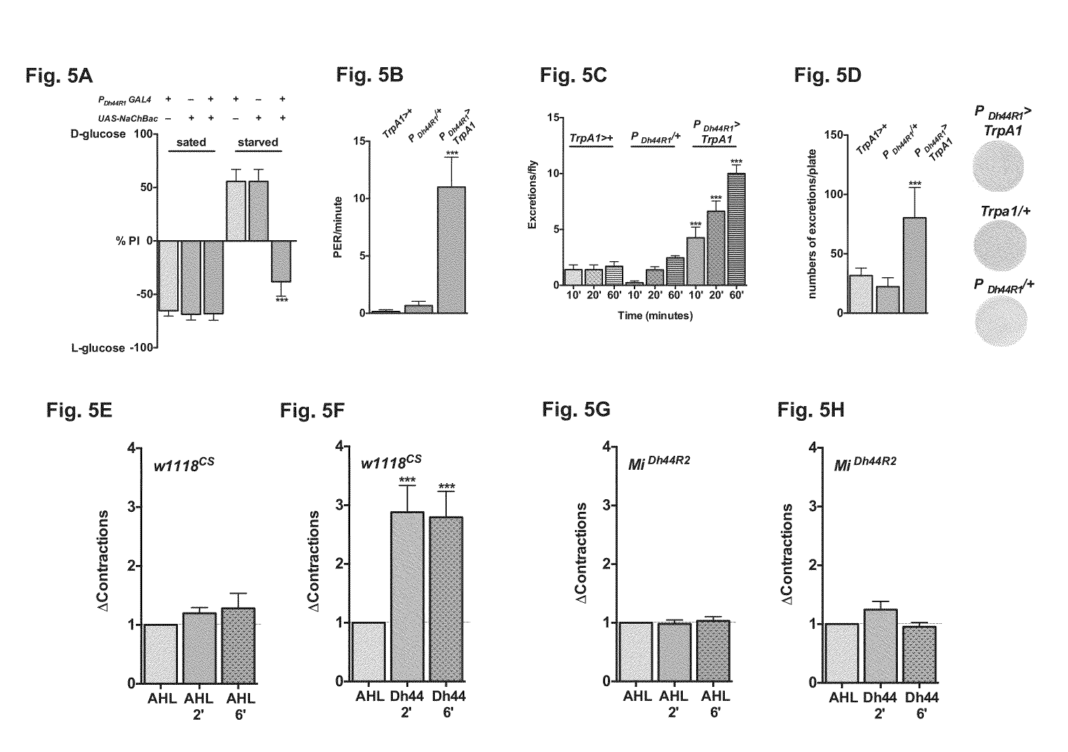

To better understand this, we used the hyperactivating Na⁺ channel UAS-NaChBac in the R1⁺ neurons and tested its effect on food choice behavior. Activating R1⁺ neurons shifted the preference to the sweeter L-glucose (FIG. 5A). This, together with the fact that R1⁺ neurons innervate the taste area which also controls proboscis extension (SOG), suggests that activity of these neurons might provide a positive feedback on feeding that is dependent on nutritional value (from Dh44 release and binding). To test this, we acutely activated the R1⁺ neurons by the expression of the heat-activated Transient receptor potential A1 cation channel, TrpA1 (P_{Dh44R1} GAL4>TrpA1, which opens when animals are switched to 30° C.) and tested its effect on proboscis extension. Flies extend their proboscis to eat, but they do not extend it unless taste receptors on the legs or proboscis are stimulated by palatable foods like sugars. However, heat-mediated activation of R1⁺ neurons resulted in rapid and frequent proboscis extensions in the absence of food (FIG. 5B). Thus, nutrient-mediated signaling through the R1⁺ neurons likely functions as a positive feedback on feeding: flies that eat the nutritious sugar will respond to it with an increased frequency of proboscis extension, reinforcing the choice for that sugar. On the other hand, consumption of non-nutritive sugars doesn't induce a positive reinforcer, likely resulting in the fly moving away from that food to explore other choices. Intriguingly, we also observed an increase in the rate of excretion in flies where the R1⁺ neurons were activated by heat through expression of UAS-TrpA1 transgene.

In mammals CRH promotes colonic motility via signaling through the Dh44R2 expressing cells in the periphery. We asked if in the fly Dh44 also increases gut motility in a manner dependent on the R2 receptor. We gently dissected fly guts in saline (AHL) leaving the head capsule intact and filmed their spontaneous contractions (in saline) before adding the Dh44 peptide (FIG. 5E). Addition of the Dh44 peptide at a concentration previously shown to activate the R2 receptor, induced a ˜3 fold increase in gut movements in control flies (FIG. 5F, after 1′ or 5′ in the peptide solution) but not in flies mutant for the R2 gene (FIGS. 5G and 5H). Therefore, the effect of Dh44 and the R2 on gut motility seems to be a conserved feature between mammals and insects. What role gut motility plays in reinforcing food choice is still unclear, as the R2 is expressed in different cell types in the fly gut. However, a better understanding of the functions of different cells types in the Drosophila intestine than that we currently have is required before we can address this. Nonetheless it is interesting to note that a large number of feeding related peptides, such as ghrelin, orexins, NPY, etc exert an effect on gut motility too.

Our work on Dh44⁺ neurons, the Dh44 peptide and its downstream signaling sheds light on both the sensing and the effector mechanisms that underlie an animal's preference for nutritive sugars over non-nutritive sweeteners. Neuronal glucosensation has been a widely studied phenomenon since the advent of Jean Mayer “glucostatic theory” in the late 50 s; however, whether glucosensing neurons function in feeding was still unclear. Our work assigns a physiological role to neural glucosensation in food choice behavior. On one hand, central glucosensing mechanisms that rely on metabolization of sugars rather than on their entry, provide a way to discriminate compounds based on their energetic properties. Furthermore, because secretion of the Dh44 neuropeptide is triggered by sugar metabolism-mediated activation of the cells, it also provides a way to communicate to downstream circuits/tissues the availability of metabolically-important sugars even in the absence of taste or in the presence of conflicting taste information (for example, bitter compounds mixed with sugars). An interesting idea is that activation of Dh44⁻ neurons and/or release of the Dh44 peptide might be affected by the energetic-value of sugars, opening the possibility that this signaling pathway could also be involved in the selection of different ‘qualities’ of sugars. Finally, effector mechanisms downstream of the Dh44 peptide both in the brain and the periphery work to trigger a positive post-ingestive reinforcer on the selection of the metabolically favorable food by increasing proboscis extension and promoting gut motility. A simple way to think about this, is that nutritive sugars start a positive feedback loop where the fly will be more likely to choose them, while ‘making room’ to accommodate increased ingestive demands due to fasting.

The CRH signaling peptides link behavior, hormonal and autonomic responses to the environment. Since its discovery 25 years ago, CRH has been implicated in stress-responses and innate immunity; however its release, functions, and signaling mechanisms are still largely unclear. Fasting is a generally accepted form of environmental stress, so it is perhaps not surprising that in both mammals and insects CRH/Dh44 have a conserved role in feeding. Data provided in Example 2 indicates that mammalian CRH⁺ neurons are glucosensing. Therefore, these neurons can also contribute to food preference for nutritive sugars.

Other Results include:

Activation of Dh44R1⁺ neurons results in proboscis extension in P_{Dh44R1} GAL4>TrpA1 flies. This was observed in the following way. P_{Dh44R1} GAL4>TrpA1 flies were introduced into a glass Pasteur pipette and left at 30° C. for 5′ then filmed with a high-speed camera at 2 frames/second for 1′.

Activation of Dh44R1⁺ neurons does not result in proboscis extension in control TrpA1/+ flies. This was observed in the following way. Control TrpA1/+ flies were introduced into a glass Pasteur pipette and left at 30° C. for 5′ then filmed with a high-speed camera at 2 frames/second for 1′.

Exposure of wild-type fly guts to the Dh44 peptide promotes gut propulsivity. This was observed in the following way. A gut of w1118^{CS} flies was carefully dissected in AHL and pinned to a sylgard plate and imaged in AHL for 5 minutes at 2 frames/second. After 5′ the Dh44 peptide was added and the gut imaged for 10′ at the same frame rate. The video showed activity of the gut at AHL-2′ and Dh44-2′.

The Dh44 peptide has no effect on gut motility in Mi^{Dh44R2} guts. This was observed in the following way. A gut of Mi^{Dh44R2} flies was carefully dissected in AHL and pinned to a sylgard plate and imaged in AHL for 5 minutes at 2 frames/second. After 5′ the Dh44 peptide was added and the gut imaged for 10′ at the same frame rate. The video showed activity of the gut at AHL-2′ and Dh44-2′.

It was also observed that sugar perfusion induced robust and sustained activity in P_{Dh44} GAL4 >UAS-GCaMP3.0 flies.

### EXAMPLE 2

This example describes experiments for electrophysiological investigation of CRH neurons in mice and show that corresponding CRH neurons in mouse brain slices also respond to glucose.

### Mice

Mice in which Cre recombinase is expressed in CRH neurons (CRH-IRES-Cre mice, Jackson Laboratory, were mated with td-tomato reporter mouse (Jackson Laboratory, #007908) to visualize the cell bodies of CRH neurons. The mice used for electrophysiological recordings were housed in a light-dark cycle (12 h on/off; lights on at 7:00 a.m.) and temperature-controlled environment with food and water available ad libitum. We used either male of female from 3 weeks to 5 weeks of age.

### Electrophysiology

Whole-cell patch-clamp recordings from CRH neurons in paraventricular nucleus (PVN) slice preparation and data analysis were performed as previously described (ref).

Briefly, 3 to 5 week old of either male or female mice were anesthetized with isoflurane and transcardially perfused with a modified ice-cold artificial CSF (ACSF) in which NaCl was substituted with an equal molar concentration of sucrose (described below). Then, the mice was decapitated, and the entire brain was removed and then immediately immersed in ice-cold ACSF (220 mM sucrose, 2.5 mM KCl, 5.0 mM MgCl2, 1.0 mM CaCl2, 1.0 mM NaH2PO4, 26 mM NaHCO3, and 10 mM glucose), saturated with 95% O2 and 5% CO2. A brain block containing PVN was mounted on a stage and coronal sections (250 um) were cut with a Leica Vibratome. The sectioned slices were then incubated in oxygenated ACSF at 34° C. for at least 1 hour before recording. The slices were transferred into recording chamber and bathed in oxygenated ACSF. The solution flowing the recording chamber was consists of: (126 mM NaCl, 26 mM NaHCO3, 2.8 mM KCl, 2.5 mM CaCl2, 1.25 mM NaH2PO4, 1.2 mM MgSO4, and 5 mM or 2.5 mM glucose). The pipette solution for whole-cell recording was modified to include an intracellular dye (Alexa Fluor 488): 120 mM K-gluconate, 10 mM KCl, 10 mM HEPES, 5 mM EGTA, 1 mM CaCl2, 1 mM MgCl2, and 2 mM MgATP, 0.03 mM Alexa Fluor 488 dye, pH 7.3. Electrophysiological signals were recorded using an Axopatch 700B amplifier (Molecular Devices), low-pass filtered at 2-5 kHz, and analyzed offline on a PC with pCLAMP programs (Molecular Devices). Recording electrodes had resistances of 4.0-6.0 MΩ when filled with K-gluconate pipette solution.

As the cells to be targeted illuminate red fluorescence from td-tomato, the pipette with positive pressure was attached to cell membrane, which was subsequently giga-sealed by brief negative pressure, before making a tiny hole with more negative pressure to perform whole-cell recordings. Only cells with a whole-cell resistance below 15 MΩ and with a stable resting membrane potential within 1 min were selected and carried out recording. First, the recording solution with a 5 mM concentration of glucose was perfused to the recording chamber and the membrane potential was recorded for 1 min. At the end of a stable trace, its input resistance was assessed by measuring the voltage deflection to hyperpolarizing rectangular current pulse steps (500 ms of −10 to −50 pA). After 1 min (when the membrane potential become stabilized after the hyperpolarizing currents), the recording solution was changed from 5 mM to 2.5 mM glucose and the membrane potential trace was recorded. Depending on the cells, it took 1-5 min to change the membrane potential and firing rate. After stabilizing the change in electrophysiological response, input resistance was assessed in the same way as previously described. To evaluate the recovery of the response, the recording solution was changed back to 5 mM glucose and the membrane potential and firing rate were measured. (For most CRH neurons as GE neurons, it took more than 10 min to recover after the response.)

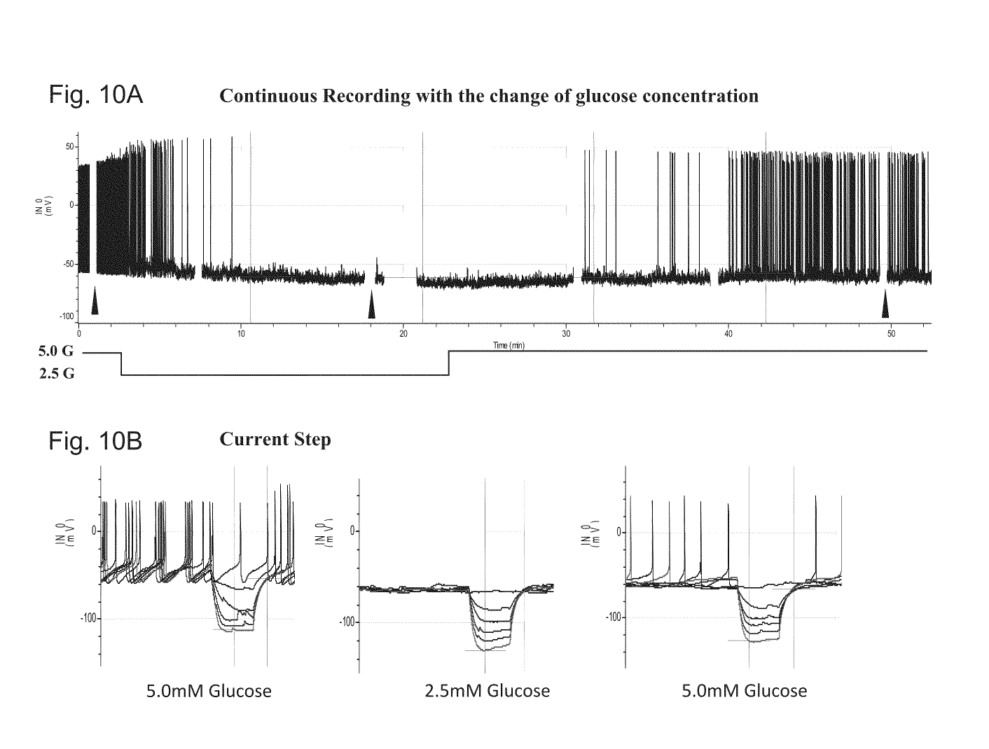

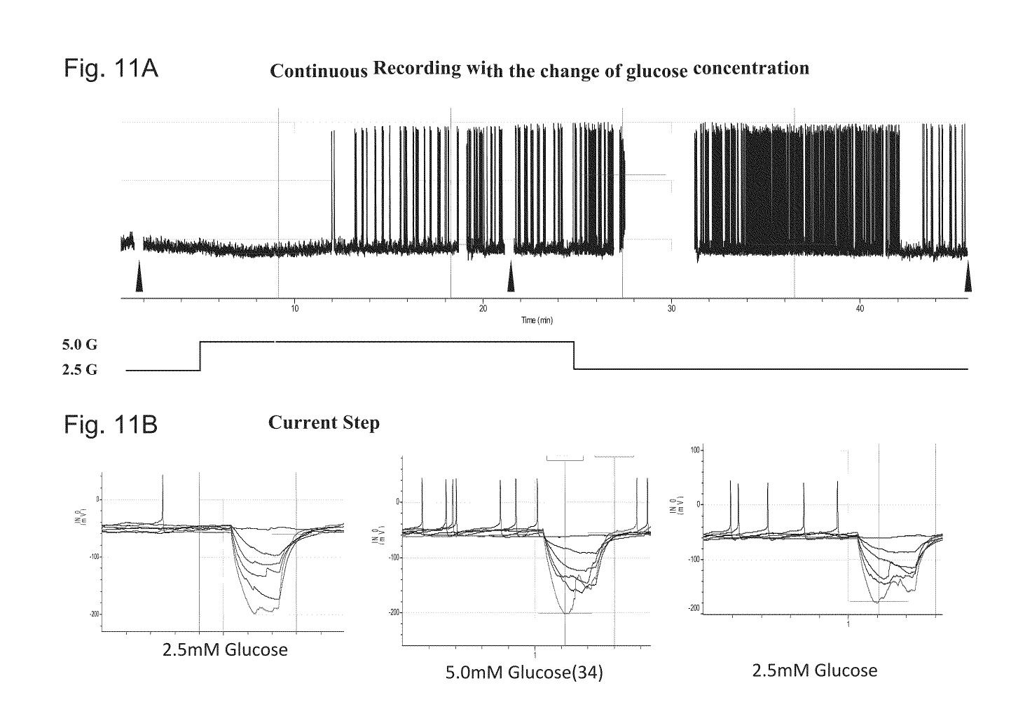

The results are shown in FIGS. 10A-10B and 11A-11B. FIGS. 10A and 10B show that the electrical activity of the CRH neurons is decreased when glucose was changed from 5 mM to 2.5 mM and then increased when it was changed back to 5 mM. FIGS. 11A and 11B show that the electrical activity increased when glucose concentration was changed from 2.5 mM to 5 mM and then decreased when it was reduced back to 2.5 mM again confirming that the effect is concentration dependent and is seen at 5 mM but not 2.5 mM. A summary of the various parameters is provided in the table 1 for FIGS. 10A and 10B and in table 2 for FIGS. 11A and 11B.

TABLE 1
Glucose Sensing Response

| | Glucose (mM) | | |
|---|---|---|---|
| | 5 | 2.5 | 5 |
| f (HZ) | 0.64 | 0.02 | 0.65 |
| RMP (mV) | −49.5691 | −65.8874 | −57.9124 |

| | Glucose (mM) | | |
|---|---|---|---|
| | 5 | 2.5 | Rev P |
| Input R | 1.40 | 1.25 | −222.95 mV |

TABLE 2
Glucose Sensing Response

| | Glucose (mM) | | |
|---|---|---|---|
| | 2.5 | 5 | 2.5 |
| f (HZ) | 0.64 | 0.02 | 0.65 |
| RMP (mV) | −60.5911 | −52.4561 | −59.3806 |

| | Glucose (mM) | | |
|---|---|---|---|
| | 5 | 2.5 | Rev P |
| Input R | 2.82 | 2.66 | −169 mV |

The present disclosure includes polynucleotides and amino acid sequences which comprise or consist of any of the sequences described herein. All polynucleotides encoding all of the amino acids sequences are included in the scope of this disclosure. The polynucleotides include their reverse complements and RNA equivalents. The disclosure includes all contiguous segments of the polynucleotides and amino acid sequences described herein, from two amino acids or two nucleotides, up to and including their full lengths. Additional nucleotides or amino acids can be added, and conservative amino acid substitutions, insertions and deletions, can be made and tested to determine whether or no they affect the function of the protein in an undesirable way.

The Dh44-RA, Dh44-PA and Dh44PC are three different isoforms/propetides.

The sequence of D. melanogaster Dh gene and the encoded peptides are provided in Cabrero et al., 2002, J. Expt Biol., 205:3799-3807, which sequences are incorporated herein by reference. The human homolog of Dh44 or the CRF gene has the amino acid sequence under GenBank no. AAH11031.1, Jul. 15, 2006, entry, incorporated herein by reference.

While this disclosure is illustrated through specific examples and embodiments, these are not intended to be restrictive and those skilled in the art may make routine modifications, which are intended to be included within the scope of this disclosure.

1. A method of screening Drosophila melanogaster fly mutants to identify mutants with altered calorie sensing behavior comprising:
a) providing a plurality of sets of Drosophila mutant flies, wherein each set comprises a plurality of flies having the same mutation;
b) starving the mutants for a period of at least 5 hours;
c) providing access to the flies to a food choice between metabolizing sugar and a non-metabolizing sugar, wherein the food choice comprising the non-metabolizing sugar is at least as sweet as the food choice comprising the metabolizing sugar, and wherein the ingestion of metabolizing and non-metabolizing sugars by the flies can be measurably detected; and
d) if the flies within a set do not show preference to metabolizing sugar over non-metabolizing sugar, then identifying the mutation as affecting calorie sensing behavior.

2. The method of claim 1, wherein the metabolizing sugar is D-glucose and the non-metabolizing sugar is L-glucose.

3. The method of claim 1, wherein the metabolizing sugar is present at a lower concentration than the non-metabolizing sugar.

4. The method of claim 1, wherein the mutants are starved for from 5 to 24 hours.

5. The method of claim 1, wherein the Dh44 gene of the Drosophila melanogaster has been replaced with human homolog Corticotrophin Releasing Factor gene.

6. A method of identifying agents that interfere with calorie sensing behavior comprising:
a) providing a plurality of sets of Drosophila melanogaster flies, wherein the flies exhibit a preference for metabolizing sugar under starved conditions;
b) starving the flies for a period of at least 5 hours;
c) providing access to the flies to a food choice between metabolizing sugar and non-metabolizing sugar, wherein for each set, at least one test agent is provided with the food choice, wherein the food choice comprising the non-metabolizing sugar is at least as sweet as the food choice comprising the metabolizing sugar, and wherein the ingestion of metabolizing and non-metabolizing sugar by the flies can be measurably detected; and
d) if the flies within a set do not show preference to metabolizing sugar over non-metabolizing sugar, then identifying the test agent for that set as an agent that affects calorie sensing behavior.

7. The method of claim 6, wherein the metabolizing sugar is D-glucose and the non-metabolizing sugar is L-glucose.

8. The method of claim 6, wherein the metabolizing sugar is present at a lower concentration than the non-metabolizing sugar.

9. The method of claim 6, wherein the mutants are starved for from 5 to 24 hours.

10. The method of claim 6, wherein the Dh44 gene of the Drosophila melanogaster has been replaced with the human homolog Corticotrophin Releasing Factor gene.

11. A method for identifying agents that affect calorie sensing behavior comprising:
a) providing DH44 neurons of fruit fly;
b) exposing the neurons to metabolizing sugar in the presence or absence of one or more test agents; and
c) determining if neuronal response to the metabolizing sugar is affected by the presence of the test agent and if the neuronal response is affected, then identifying the agent as affecting calorie sensing behavior.

12. The method of claim 11, wherein the neurons are exposed to caloric sugar in vitro.

13. The method of claim 11, wherein caloric sugar is D-glucose.

14. The method of claim 11, wherein the Dh44 neurons are provided in isolated brains of fruit flies.

15. The method of claim 11, wherein the Dh44 neurons are provided as cells in culture.

16. The method of claim 11, wherein the neuronal response is release of a Dh44 peptide.

17. The method of claim 11, wherein the Dh44 gene of the fruit fly has been replaced with the human homolog, CRF gene.

18. The method of claim 11, wherein the neuronal response is release of CRF peptide.

19. The method of claim 11, wherein the neuronal response is increased electrical activity.


SEQUENCE LISTING (7 sequences)

SEQ ID NO: 1 - DNA, 1067 bp, Drosophila melanogaster
atgatgaaagccacagcgtggttttgtccggtgttattaactttactgtgtgccacaaggctcgtctgcacggcccaaagaggagcagttggaacaggaggagctgcaggaggatcgggagcggcagcgggtggagcggaggtcggcggaagcggccgcacaaacagatatcctctggactacccggatggaacacggaactcacaggatgactttctgctggccaagaggaacaaaccgtcgctgtccattgtcaatccgctggatgtgctgcgccagcggctgctcctggaaatcgcacgcaggcaaatgaaggagaactcccgccaggtcgaactgaatcgcgccatcctgaagaacgtgggcaagagggtcgtgctgcgaggaggaggaggaggcggaggatcgggagcaggaggattggctcccaaggtgagcaggcgctaccgccagcagtggccagtggagcgggaactggagcgggaaaggcagagggagagggaaagggaatgggatgcagttcgtgaggagcagctggaccgtcaacagctgctgccgtggaagcatttccccagccaactgtggagctacggatgggccctgtcgccgtacaaggagtcgtcacagttacagtttgcggactcccagcaatcagcatcgactggaccacaatcccaggcactcccaaagcagttgcagttgctcagctatgcgaagaaaccactggacgtggccggaatgagtctggccaggcatcgggtcagcggaaatgaagccaacgaaacgaatcatgagaacgacgatggcaatggggccagtaaaaacccggcgagatatgtggatgacggcgacaatgagggcgaggatagctacaatgacgtgggaacagaaggtgtgggactcggcctgggaatgggtgtgggattgggtctggaacggttcgaggtgctggaggacaagcccaactgggccgatgaggaaccaaacgagttggtggtggttaatgcaaatgaccgcgtgccctggtcctttccctaccgctttcacaaaagtcagcataacgttaa

SEQ ID NO: 2 - protein, 356 aa, Drosophila melanogaster
MMKATAWFCPVLLTLLCATRLVCTAQRGAVGAGGAAGGSGAAAGGAEVGGSGRTNGYPLDYPDGTRNSQDDFLLAKRNKPSLSIVNPLDVLRQRLLLEIARRQMKENSRQVELNRAILKNVGKRVVLRGGGGGGGSGAGGLAPKVSRRYRQQWPVERELERERQRERERERDAVREEQLDRQQLLPWKHFPSQLWSYGWALSPYKESSQLQFADSQQSASTGPQSQALPKQLQLLSYAKKPLDVAGMSLARHRVSGNEANETNHENDDGNGASKNPARYVDDGDNEGEDSYNDVGTEGVGLGLGMGVGLGLERFEVLEDKPNWANEEPNELVVVNANDRVPWSFPYRFHKSQHNVN

SEQ ID NO: 3 - protein, 356 aa, Drosophila melanogaster; identical to SEQ ID NO: 2

SEQ ID NO: 4 - DNA, 12525 bp, Drosophila melanogaster
ctaatatctaccaatttatagaacgctctggtatgtaaacatttccattgaactgggagaaattcgcaataaagcggttcagatagtggaaaatgggttgaacaagggaaagttttgatattttaataaaatcatgtataacataggaaagcggttttcgtaattaccaaaaccatgaaacaatcaatactgaaatacaaaaatatgttactatcaaaataatggcttatacgattggcctaaggctttattaggtcaagcaaattaatattacttgcttacctttcgttaattttatttgctgaagagcgtcgcaaatgtcaagaagtctagcaaattactagaaattacataagtgtgtttcgtttgcagtaaattcagtaagtcgaaagcggctcaaagatgaaacaagttggaaaaacttttctgggattagccgaaatgtatattggtcaaactttttggctaacttctgctgctgtttgatttttgttcgctttgcctaaatggtccgtaacttgatttaattgatgtgcttcaaagaataatccgctcacttagtcgcgtttcaaagataaccaactggaagccaaatattgcggtcgaaaaacttgataacactgttaattgaatgttttgaaggcggcgtgatgtccctctttaataatcaataaagacattcctaattattaatgattaaacttgaccgaaaccaagttccctgacactcacacaatcgtcaaatcgcgcataattggatgggaccatcggctgcccataacaaggataattggatgaaatgaggcggctgccttttctgctcctcattaaatgcccgtttaccgtttagtcgcctgtcaaatccttgatgggctattagaaaaaggggtgtccagagacgagttcttgagtcattgacttgttacgcaaattggacaaaagaaatttctggctccgagtgggcactggagtggggattgaacttaataagcatattcattaatgggtgtacagaaccaagtacaaggttgcatcttacacccttatggatatcctttccttcttgaatccctaagcaaaagtaatcaggcaagtagctagactttactagttgctctattaaaaattaaaagtaaattaaaattgcctcgcactaccaaaacagcattacagctcgcataaaagcacgtataaattggcgggagtggcattttgacggaattcaatcagccataaagcatcctgctgggcaggaaatggaggcgtttgcctacgtgcttaattacaagccgacgcatgcacattctgcatacataaaaggcgaagacacgtgttccaagcaattaaaaagtatgtcaaagtgggtgatcaacgcgcccaaggacgcaggacaattgcgaatgctggcaacactgttgctttttttgagtgatgcgtgggctagccaaactagttgatgtagatggcatgaaaagtttggggggtcccaaattataaattgcttatctggctgtgtgtacaatgtggatagcaatgtgatataaatggtaattgaattcgttttcaaggtaatcagcagtaatgattgctgcttttcttagcctaaatagaaaggatatcggcctctgttagccttcgcatatcccaataaaaattgattggtccaaaataagacgaaacacccagcaggcagagcacctcctgcgtggaatccatctcctggccagggatccctgtggctaacgtttgacggatttatggcgagcaatttaacgcccgattgccgtcctagcggaatgtcgggccacgttagcacctcttgacatgacatcgagtgctccggagtccgcatcctcgtcacttttaaatcgaacaattaatgtgcaaataaaactcattaaatattaattgagttcgataattacagagttgacggggttgcggcttcgcgcaacatgttgctgaagcacctgatttgtgggaggtcaccatcaccacttcgaaaccaaggggcttcgggtcacccactggcaacaatccgtcaatttcctaatgcgttcgcacaccctggtgatttattagtgaggattgcagaccgaaacgacgcggtctgacccgcctcatctgattccggagcgatttccatcatcctggcaacatgccgtacccgcacgttttcattcataactcgtcgcgtggcttcagcaccttcctttgccttccgagttcttcgggctcccacatccgtcgtcctgtcgtcctgacgcctcggacttcgtgctcggatttcggatccggcacttcgtgtgggcgcctcgtggcgcccaatataaaaacacgccgtaatcgctatactccgtgagtcctccgctcgtcgtcagcctgttgcgttcgaccctcgatttcgatgctgggtgaaaatccatcagaggaaccaaagccacaagctacagcaacagttcaagattccgaatcagttaacgtttcgcgacgcgacgcgtgaatacataaaataaattagaaaaaagataaacaagccataaaagtaggggggctctttcccaaaataagacaattcgaaaacccacgcgaaatatccagtatctcgatcgtcataaatcaatccgtcgagtgtgcatccgagttccgcagatccagcgaaattagaggcaattaccactgataaggtgatttcttcttttatgtgataattttgggaaagagtgctgtttctatgtttttgagtttggtgtttagtgtttggctttttggcgaaaacatatcagatttcgggcgaaaacctcaacggaaatgtggcgcaaattgaatggcccaaaaggtggcggcaagcttgattgatggcccgaagaacgtttaggctaattagttttgcattttgtgttcggtttcaggtaacaacacaccgacgaaacctaatagactgcaaacaagcttagcgaaaggtcggcaatcacacctggtaattttctttgctccacccctgagctgcagattcccaaatgattggccttttgtgctgatttttttacggcctaatcaaatgggatttacggcctttaaagcggccagagctggaaaacttttcacatactcgtatttgcatacgcatgtaaatatctcgagttggtggcatagatagcagggatttcatttccttgtgccgcggtccgccctttgaattgccgataaaacaagaagggacgtcctggcaagtagccctgggcaatcacgatttccgtctccgagtcccagagtcgcccagtaaatgtatgacaatggctgtcaggagcacaatcctcagtggggcagggcagttttattgccttgaattccatattactttcacaaaatcaatatttgttcaacttcaaaagtaagaatgtaagaaaattcattcattatatcggctggggaatactactcactcccctcgggatccctatttcaattggattgcaatttccgttgcaacttcgatggttgttaccaggaatagattcatcacatctctctccagcatttaggcgatctattaggggaaatcaatagccatcaattgtcgtttgccgaaagtgccagcaacccacacacccacggatttccaccggattcagatgcggattcgtaaatccccgttcgccgaatggcatccatatgacatgtgattgccggcatggactaatgcggctgccattcgagcacttagttatctgcgatgtctaatgctcccgatgacaggtgctatatgagctgtatgtcccgttccccctcaccgaaaacaaaggagccgacttaattcccaaaatggggaggttttcatagtgcaactttgagttgctgatgagttgccgaaaactcatgtgtaaatttcacttgcacagagtttgtaatgaaattggtttctttttaaaaattgccaatttgtgtttaactttagagctgccatcgctcttgggattataaattatttaggaatgttaaacctaattttgtgtaatgcagaaatttgagttatggcagttcaaacttttacgtacgcacacactaaattgcaagaagttttccttgttgtggcaattgttacttaattttgttagctacacaaagttacttggattagggaactttcattttgagaacaatggttgctttacattgcgtcggtgtgggaaaaccaaagtgaaactttcagttgttctacgcaattaaaagtgacctcatgaaaggattatgatggaaaaccaggggaaagaaaaatatgttaaataaatatgggagaaactgaaaggaggacaaaaaaaaaaaaaactcctgaaagcgccaaagggttttcatttaattatgcatttacacgggtttgtttatagatcaaaagtaaaaaaaacgttcgtttatgttgaattaattggtgaatgctcttccagggattactttagactttgaaaaatcacctatttgtctgcggctaagaaaatgaaacaatctaatagatacatgatactctagtagagtatcttcatgggctgttatgtgcctaaagtatctttgaactttttctttccaagttaagcaccttttcctctttttggaaataacttttcccctggtaactttagctgtgcaacaggaaaataatctagttgtgctgccgtttactcgtcgccgtcttcaatttagaacctaatttcctctgtgcacctcgtgcaaattgcccccgactccagttgccaactgattgaagagtccagagggacatgggattgatatacatacatatgtaaatatataggtgcatactcggaagtacataaagaactacactgccacattcagaagtaccacatatttacgtgacccataaaatatgagagcacttcttgttgtcagaatatttcgcatttttcacaggttagctgtcagcattttattttcactctattccagtgggaaaacggcggaggtcaaagtttgcacaagacagtggctcggaaaatattcaaaaaataatagccacagatgagcttaattttccacgcaaatgaacagatcaaatttgcatagcgtatggtactttgcataccctataacctatagtactgtaaataaatatgcatagttttagcagagttggttgatatgatatatacactaatactcacgttcttcattcattttgatagatttttatttatttttttatgctgaaactgttggcgttacatgcatatctatgaattgtgcactgacccatgcaactgtgcgtttttttttctcctccttttttgcagaatgatgaaagccacagcgtggttttgtccggtgttattaactttactgtgtgccacaaggctcgtctgcacggcccaaagaggagcagttggagcaggaggagctgcaggaggatcgggagcggcagcgggtggagcggaggtcggcggaagcggccgcacaaacggatatcctctggactacccggatggaacacggaactcacaggtgggcatgggcatgggcatgggcatagtcctggccattggtgtgcatttctttctggctgtctccctttgtgagtgagtgacagcagtttttgcgtcacttgtttgaatgttttccgaatgcatattcaattattggctcggcagttcgcaaacgggcgatttcatcgctcctttgacaaccttcattggttccattcactcggttcataaattgacacattgttcgccacaatgcgttaacttttacaagaagccagaaaatgtgaccgcaaagggttccgcacaaccctttaaaaccaaatagattgaaaaatatctcatacgcagtttatttagcgcggatatattaaattgctaaaagattaagctcttatcaagagttatccatacatgggaagtacattaggcattttatagtagtggcaaatcttcattctccgatttcctcgaaatcaataccacattattggtacctcgatctgaattttggccacaatctaatctaggcacttgaccaattcgaatacctcaatgtaaaacttcttcggtcgaacaaaaagatcgtataaattcccaaacgcacacgaacatggcaacaaaattgatttgagtctatgggttttttatgttttaaaaaacaaatggaaatttacgtcaaggaagaaaaaaagaaaaccaaacatgctgtttgtcttggccaactcctgttgtccacaatcaaaagttttaccactgctttttaagaagttcgaattttgttaagttgattctttattctgtattctttattcgctggaattggagtgcgaggcaagttcgtttgccaaattggagatttaagcacgtaaatgcgaaggacacgtaaacaatttaattttatttactgaaagaaatgaaatgatgttttatgatgggggcagaattaaaaatacaaccccgaggcgacgttaaaggccaattacgaaattttccatttgtaccaccataaatgacacatcgttgggcacaaacatatgttctttcgaacaaatgggcatgtgtacatacatatgtatgtttcgataagggtcgattggccaactaatcagtcaagtagcgattcattgattgatgtatgtacgtcaatcaatcagtttatcatcggctcgcaacgcttaaaaagccaatgttcatggttcggaaagcgaaagttcaattaagtgataagcattataatgcgtttagaaccttaaagttttgagtaatgtcatataagtaaacgtgacttgggattttttcatttttcggttgttttaattgttcagattgttggagctataaaactaaatggcgagtgcgtgacataataacacgatgtgggttcatgaggcttcaatcctaaccctagaaatggttttacgctcttctgggagaacatgttaataatttagctataacagtttaatatttgcaccatatttcgttttataacgctttgtttaataaccttgttactcggaaacgccaaattttaaacgattattgagaaaatgaaccagaaaccttggtaacaatgaaactaacttcctagaccgctgaagaatcgatagtggattcttccggcgcctcacttgtggattcttcgggagcctcggtcgtcgattcttcgggtgcctcacttgtggattctacaggcgcctcggttgtcgattcttcgggctcctctgtggtggattcttcgggcgcctcgctggaggattcttcgggagcctcgctggtggattcttcaggctcctcggtggtggtttcttccggagcctgagtggtatcctcagaaccgctaccatcctcagagtcggtggtgctttcctccggtgcctgagtggtggattcctccggaacttcaggggccagggtggtggtgtcctccgaggaaccgctgccttcttcagagccggtggtgatttcctctggtgcctgactggtagattcttctggaacctcgggtgccagagtggtctcctccgcgggactggtaacatccgtggtggataccacatcatcctcggtggtcaactcaggcagatccgttggtggtggtggaggagggcgctgtccaccgtggccttgaccattaccatgaccatgactgttgccatgatgaccatgatggttgtttccgtgtccgtttccttggccgaggccttgattgtttccgtgagattgtccctgaccattttcgccatcccactgaccctgaccgttgccatttggattccactgattttggctattccacctctgttcctgcacgatttcatcgtcaccggagccccaaccgaacctcctggcccagttgctgccaaaatttccgaaccaaccgccattggggcggccagaagccactgccaccaaggcagtgaagaggataagagttaggaccttcatttcgctattgtttcgattatactaccgggttctgcagatggacatagcttttatagaaagctgataaccacttccattcactgccagacatttcgacctcacactgccacattcttatctaatcgcaggtaacctgtctgatagtcagaccaacaataatcgtaattatcatatttatctgaacatgaattcggattggttgtttactgtttgccctgaacttaggatacgtaaatacttcttggaaggaacaccaacaaataaaagataatatttaaatttttccaaagaagcacctgtccagactctgtataaatttcgggtaatttctttatgatcaagcaaaatccaaatcccctttttcatgtggtgtaaagaaattgcgatatgcgttgttgggcggatcgtaaagtgatagtatcaggtgttttttaaccgcccccccaaggtaattgtttttgaattttattttgtggctacgtgtgagtcatgaattagcttgaagttttcacacttatcagtgaagagctgttatcacgaaattcccaattgatcatcgtggaaaaaacgtagaacaacacaccgataagcttgcaagattttgtgtaacgataatttcccgaattgtgggaaaaacaaaaggtgatataaaaaatatgcgatagccgaggcccttaagattgctcaccatcgatgctaataataattttaatttccatcaacaaacaaaacaaaaaaagaataaacttcgagcacagaactgtatataaacacagggacttaaaactttttgcattacctcgtcaggattttattatgccatgcgctgggaaacttcttccagccaaacagcgaatctaacgtactaaatatgtatgtatttcgttctaggatgactttctgctggccaagaggaacaaaccatcgctgtccattgtcaatccgctggatgtgctgcgccagcgtctgctcctggaaatcgcacgcaggcaaatgaaggagaactcccgccaggtcagtctcgtgttctgcaatattaaacggggatggctgaccacttggccaagtatttttaataagctgcaaaggagggtaaaacaaaactttggcttcgtatggcccagacaaacggagaacaatgccgcgtcttaaggggaatgaaagtaggtcgcgttgcaaggtataaatgaaaatgtagcgggctaatcaaaactagaatgcctgatgagaaatacttttcgaaagccataaaaacgcataaaacgcaggtgcagaaagttttttaattcatcgcagctagcgaatctaataaatttccaaaggacccaaatggtaagactctgtgcagcccacacacctacttgctcctggaatgtaaacaacatcctggcgaacccgaaatcgaggcttaagcacatcggatggccctgacacccaaaaatatacatacgtatatgcatgcggctcagtgccaaaaacaaagccaacatggcgtatgtgtaacgcaaatatttttgctcggtaacaaagcgagtttatttggttaccaaaacgttccgatcccccaaaggcgcacacacaaagacaaaaaggccaacgaaaatgttaaaaacaaaaaaaatcaaacgttttaatacgaggaattcaaacgatttgccttgatagcttcacctttcctcccacgaatttccgttaaagagggtcatgttcatccatggatgtgcctatccgcatgcccacctacctattcacatacgccagataacattattgatttgtcaaaggggtttcataaattaatccatgcatcccgaaagcaatccgaatcggagaaagatgagcttcagcaggcataatgttttcggaaaacacctgggttagactggcaaataaaagggaaataaaatggagtgaaattaaagtgaaatttcaaagaaacaaaatgaggttttcaacggcaagctgtcaaatttaataccacacaattaaaggaaagttacatttagaaacgtgtatttaaaagttaggatggagctagatatctgaaatagcattcaccacttgaatttgaatgcaaacaaaatgaatcgacatacttattcacagcttaaaggaatagatcacgtttcggtttgtgcactattcccagcagatgaaaatagactttcacatggccaaaaatataaagggaaaaatgcaatgtcacccgaacttacgagttagacattgaatccccagctcaatgcaaacacctgactctttaaaagacctgcaaagtggttggcaaacagaaagaaatttaaataaatatccaaagcactagaagaatggaatttccaaggtgtgaaaactgcaagttggcatttacacttaaattggatttaagctaaaccgagtattaagttaaaagataggaaaaggtataaatacatttccaacgattaatcatcttggctttaaatattaaagtatgttttatacaagcacctgagacttaaagtaactataaagtaattaggcaaagtatctggtggcttttcagtttagcaactaaagctgctcctaaagtatcttaaagtgcttaaaatgtaatcgtgccacatacctcatcctcgattaatgtaataacgcactggcatttgatttgtgatccaaattgaatgtggtcatccatcatgcatgtcaatctagtttagctttccctttgccgggaaaagtacatggaaaatccatggggataagggagaaccgggcttaccccacttccatttcaatttgtcaaccattttacagcagttaacagcattttactttacttaactttatgtgaaccgtgttacctttcttgtgtccaggtcgaactgaatcgcgccatcctgaagaacgtgggcaagagggtcgtgctgcgaggaggaggaggaggcggaggatcgggagcaggaggattggctcccaaggtgagcaggcgctaccgccagcagtggccagtggagcgggaactggagcgggaaaggcagagggagagggaaagggaaagggatgcagttcgtgaggagcagctggaccgtcaacagctgctgccgtggaagcatttccccagccaactgtggagctacggatgggccctgtcgccgtacaaggagtcgtcacagttacagtttgcggactcccagcaatcagcatcgactggaccacaatcccaggcactcccaaagcagttgcagttgctcagctatgcgaagaaaccactggacgtggccggaatgagtctggccaggcatcgggtcagcggaaatgaagccaacgaaacgaatcatgagaacgacgatggcaatggggccagtaaaaacccggcgagatatgtggatgacggcgacaatgagggcgaggatagctacaatgacgtgggaacagaaggtgtgggactcggcctgggaatgggtgtgggattgggtctggaacggttcgaggtgctggaggacaagcccaactgggccaatgaggaaccaaacgagttggtggtggttaatgcaaatgaccgcgtgccctggtcctttccctaccgctttcacaaaagtcagcataacgttaattaagtaagtagggattgagatgcaaacacagatatacagatacgataaagatacagatacagatacagatgcatatgcagttacagatatatgtaataccgacacagataccgataccgataccgaaacggatacgatgcgataggggcacccaacggcttctgagtgctggaaggttattaatgcaaatcgacggttcctaatggctggctacaaggcaggcatgttgtgtgtgcacatactcccttgatgacaaacttactcccacgttgctagatgtactcgcatttgaactctattttatcgaaaataaccaacggtgtatgtaacgatttatgctacctctctcacagatactgttgaaaataaagtccccgattaatgtgaaacatacatagatacccatgctaacgtgcatactactagatactttactctagatcgaaatggccgaaactttagccaatacaccaatgctgctaaggattataaacgtaattgcatgtatgcgtaattgatagtgatctatgtgtaatttaaaaaattgttttaacgctcgacgttaattaatcaatcgcatgcaatattgactagtcaaatgggattttcacgtttttatccacataagcaaaacttgtttaccaactattaattgtggcttacattctccgcgggcggaaatcggtttttatgttcattacgtttgcttcgttgtgctttaacttgtgcaaaacaaaacaaaaaaatgctcaaatagaattaacatagtgccgaaagtactgttcacacactcttttcgttaaagctgcatttggtcactattgaatttgcgctgttgaagggttcaagtttaaagctatcaggcgacaaatggttccaaatcatacgagtaacgagtaacgaattacaaattacgaataactaataacaaattgcaaataacgattttaacgtagatcaaaatcagaaatattacccaagcaagt

SEQ ID NO: 5 - DNA, 1071 bp, Drosophila melanogaster
atgatgaaagccacagcgtggttttgtccggtgttattaactttactgtgtgccacaaggctcgtctgcacggcccaaagaggagcagttggagcaggaggagctgcaggaggatcgggagcggcagcgggtggagcggaggtcggcggaagcggccgcacaaacggatatcctctggactacccggatggaacacggaactcacaggatgactttctgctggccaagaggaacaaaccatcgctgtccattgtcaatccgctggatgtgctgcgccagcgtctgctcctggaaatcgcacgcaggcaaatgaaggagaactcccgccaggtcgaactgaatcgcgccatcctgaagaacgtgggcaagagggtcgtgctgcgaggaggaggaggaggcggaggatcgggagcaggaggattggctcccaaggtgagcaggcgctaccgccagcagtggccagtggagcgggaactggagcgggaaaggcagagggagagggaaagggaaagggatgcagttcgtgaggagcagctggaccgtcaacagctgctgccgtggaagcatttccccagccaactgtggagctacggatgggccctgtcgccgtacaaggagtcgtcacagttacagtttgcggactcccagcaatcagcatcgactggaccacaatcccaggcactcccaaagcagttgcagttgctcagctatgcgaagaaaccactggacgtggccggaatgagtctggccaggcatcgggtcagcggaaatgaagccaacgaaacgaatcatgagaacgacgatggcaatggggccagtaaaaacccggcgagatatgtggatgacggcgacaatgagggcgaggatagctacaatgacgtgggaacagaaggtgtgggactcggcctgggaatgggtgtgggattgggtctggaacggttcgaggtgctggaggacaagcccaactgggccaatgaggaaccaaacgagttggtggtggttaatgcaaatgaccgcgtgccctggtcctttccctaccgctttcacaaaagtcagcataacgttaattaa

SEQ ID NO: 6 - DNA, 1071 bp, Drosophila melanogaster; identical to SEQ ID NO: 5

SEQ ID NO: 7 - protein, 196 aa, Homo sapien
MRLPLLVSAGVLLVALLPCPPCRALLSRGPVPGARQAPQHPQPLDFFQPPPQSEQPQQPQARPVLLRMGEEYFLRLGNLNKSPAAPLSPASSLLAGGSGSRPSPEQATANFFRVLLQQLLLPRRSLDSPAALAERGARNALGGHQEAPERERRSEEPPISLDLTFHLLREVLEMARAEQLAQQAHSNRKLMEIIGK